(19) United States
(12) Patent Application Publication
(10) Pub. No.: US 2023/0172895 A1
(43) Pub. Date: Jun. 8, 2023

(54) METHODS OF TREATING OR PREVENTING ORGANOPHOSPHORUS POISONING

(71) Applicant: LACHESIS BIOSCIENCES LIMITED, VICTORIA (AU)

(72) Inventor: Timothy Matthias MORGAN, Quincy, MA (US)

(21) Appl. No.: 17/999,013

(22) PCT Filed: Jun. 24, 2021
(86) PCT No.: PCT/AU2021/050662

§ 371 Date: Nov. 16, 2022

(30) Foreign Application Priority Data
Jun. 25, 2020 (AU) 2020902131

(51) Int. Cl. A61K 31/27; A61K 9/00; A61K 47/38; A61K 45/06

(52) U.S. Cl. CPC A61K 31/27; A61K 9/0043; A61K 47/38; A61K 45/06

(57) ABSTRACT

The present invention relates generally to methods of treating or preventing organophosphorus poisoning. In particular, the present invention is directed to use of intranasal formulations comprising rivastigmine for treating or preventing organophosphorus poisoning.

### RELATED APPLICATIONS

This application claims priority from Australian Provisional Patent Application No. 2020902131 filed 25 Jun. 2020, the entire contents of which are incorporated herein by cross-reference.

### TECHNICAL FIELD

The present invention relates generally to methods of treating or preventing organophosphorus poisoning. In particular, the present invention is directed to use of intranasal formulations comprising rivastigmine for treating or preventing organophosphorus poisoning.

### BACKGROUND

Organophosphorus compounds (OPCs), such as nerve agents and pesticides, pose a significant poisoning threat to both military personnel and civilians due to their potential use in both a direct (e.g., terrorist or military attacks) or indirect (e.g., by accidental poisoning) fashion. For example, in 2013 sarin was used in Damascus during the Syrian War, resulting in the death of at least 355 people and injuring over 3,600 people. Sarin was also used in terrorist attacks in Matsumoto and Tokyo, Japan in 1994 and 1995, respectively, causing 19 deaths and injuring over 6000 people. More recently, Novichok was used in 2019 to target a Russian military officer and double agent in Salisbury, UK, causing the death of a police officer and injuring three others. In addition, organophosphorus (OP) poisoning from pesticides causes over 200,000 deaths each year due to both accidental and intentional (suicidal) exposure. Accordingly, OPCs are a major public health hazard worldwide.

OPCs are inhibitors of carboxyl ester hydrolases, particularly acetylcholinesterase (AChE) and butyrylcholinesterase (BuChE). AChE is found in the central and peripheral nervous systems, neuromuscular junctions, and red blood cells (RBCs), where it is responsible for degrading the neurotransmitter acetylcholine (ACh) into choline and acetic acid. Due to their physical properties and high lipophilicity, OPCs rapidly penetrate and accumulate in the central nervous system (CNS), where they inhibit AChE activity by phosphorylating the serine hydroxyl group located at the active site of AChE. A feature underpinning OP toxicity is irreversible inhibition of the key cholinergic enzyme acetylcholinesterase (AChE), critical in hydrolysing acetylcholine (ACh). A large loss of AChE leads to abnormal accumulation of ACh within cholinergic synapses, resulting in the excessive stimulation of muscarinic and nicotinic receptors within the central and peripheral nervous systems. In the brain, excessive stimulation of cholinergic neurons induces the release of glutamate, the overactivation of the N-methyl-D-aspartate (NMDA) receptor, and excessive influx of calcium leading to excitotoxic neuronal cell death (Chen et al., 2014).

Current treatments for OP poisoning involve a combination of atropine (a muscarinic antagonist), pralidoxime (also known as 2-PAM, a reactivator of poisoned acetylcholinesterase enzymes) and benzodiazepines to control seizures (Buckley et al., 2004). However, atropine only works on muscarinic receptors, leaving nicotinic acetlylcholine receptors open to excessive acetylcholine binding. Pralidoxime complements atropine by working on nicotinic receptors in the peripheral nervous system, but leaves the CNS vulnerable to OP toxicity, and is not effective against all OPCs.

Pyridostigmine is also commonly used as a pre-treatment against OPC nerve agent toxicity (Sidell et al., 2008), followed by an antidotal mixture of anticholinergic drugs and an oxime, administered after nerve agent exposure. Pyridostigmine pre-treatment alone provides only minor protection against OP poisoning, mainly through enhancement of the protection provided by the antidotal injection (Sidell et al., 2008). Pyridostigmine does not readily penetrate the blood brain barrier (BBB) and therefore does not protect from organophosphorus-induced major CNS symptoms. Galantamine has also been proposed as a centrally penetrating cholinesterase inhibitor for treatment of OP poisoning (Aracava et al., 2009). However, evidence for galantamine penetration from plasma to cerebrospinal fluid (CSF) in humans was shown after an extended 3-month dosing period in patients receiving either 16 or 24 mg per day of oral galantamine (Kadin et al., 2008). In contrast, rivastigmine demonstrated penetration from plasma to CSF after a short 3 to 7 day dosing period in patients receiving 4 to 12 mg per day of oral rivastigmine (Cutler et al., 1998).

Rivastigmine is a reversible cholinesterase inhibitor that is able to penetrate the BBB (Cutler et al., 1998) and is currently used for the treatment of patients suffering from neurological conditions, such as dementia caused by Alzheimer's disease and Parkinson's disease (Birks et al., 2009; Birks et al., 2015; Maidment et al., 2006). Rivastigmine has been approved for the treatment of Alzheimer's disease and Parkinson's disease as oral capsules and transdermal patches. However, oral rivastigmine has been shown to be unsuitable for use as an efficacious and reliable treatment for OP poisoning in otherwise healthy individuals, such as military personnel, due to its poor tolerability (Lavon et al., 2015). The detachment risk, skin irritation, sleep disturbances and lower pharmacodynamic efficiency associated with transdermal patches also offer limited utility in high-performance operational populations (e.g., military personnel and emergency first responders).

Accordingly, there is a need for improved or alternative methods for treating or preventing OP poisoning.

### SUMMARY

It has now been surprisingly found that the intranasal delivery of rivastigmine may provide an improved method for treatment or prevention for organophosphorus poisoning, particularly in young healthy adults.

Thus, in one aspect, the present invention provides a method for treating or preventing organophosphorus poisoning in a subject comprising administering an effective amount of a sustained-release aqueous intranasal formulation to the subject, wherein the formulation comprises rivastigmine or a pharmaceutically acceptable salt or solvate thereof, a pH modifying agent and a thickening agent, wherein:

the rivastigmine comprises about 0.5% to about 15% by weight of the total formulation;

the thickening agent comprises about 0.25% to about 2% by weight of the total formulation; and

the pH of the formulation is in the range of about 3 to 6.

In another aspect, the present invention provides use of a sustained-release aqueous intranasal formulation in the preparation of a medicament for treating or preventing organophosphorus poisoning, wherein treating or preventing comprises intranasal administration of the medicament to a subject, and wherein the formulation comprises rivastigmine or a pharmaceutically acceptable salt or solvate thereof, a pH modifying agent and a thickening agent, wherein:

the rivastigmine comprises about 0.5% to about 15% by weight of the total formulation;

the thickening agent comprises about 0.25% to about 2% by weight of the total formulation; and

the pH of the formulation is in the range of about 3 to 6.

### BRIEF DESCRIPTION OF THE DRAWINGS

FIG. 1. Plasma rivastigmine and NAP226-90 concentration-time profiles (Mean±SD) for (A) rivastigmine nasal spray and (B) Exelon oral capsule in healthy young males (n=16).

FIG. 2. Individual Plasma rivastigmine and NAP226-90 concentration-time profiles for (A) rivastigmine nasal spray and (B) Exelon oral capsule for a healthy young male who vomited after oral dosing alone.

FIG. 3. Mean plasma BuChE maximum inhibition % versus mean plasma rivastigmine C. in healthy young males (dotted line=line of estimation).

FIG. 4. Mean CSF AChE maximum inhibition % versus mean CSF rivastigmine Cₘₐₓ in Alzheimer's disease patients (dotted line=line of estimation).

### DEFINITIONS

Unless defined otherwise, all technical and scientific terms used herein have the same meanings as commonly understood by one of ordinary skill in the art to which this invention belongs.

As used herein, the terms “composition” and “formulation” have been used interchangeably and have the same meaning.

Unless otherwise specified, the indefinite articles “a”, “an” and “the” as used herein, include plural aspects. Thus, for example, reference to “an agent” includes a single agent, as well as two or more agents; reference to “the composition” or “formulation” includes a single composition or formulation, as well as two or more compositions or formulations; and so forth.

As used herein, the term “about” means ±10% of the recited value.

Throughout this specification and the claims that follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The term “consisting of” means “consisting only of”, that is, including and limited to the integer or step or group of integers or steps, and excluding any other integer or step or group of integers or steps.

The term “consisting essentially of” means the inclusion of the stated integer or step or group of integers or steps, but other integer or step or group of integers or steps that do not materially alter or contribute to the working of the invention may also be included.

Unless otherwise specified, reference to a percentage (%) content throughout this specification is to be taken to mean a percentage by weight ( wt %). Unless otherwise specified, any reference to a percentage by weight ( wt %) of a component of a composition or formulation described herein refers to the wt % of the specified component with respect to the total components of the composition or formulation.

The reference to any prior art in this specification is not, and should not be taken as, an acknowledgment or any form of suggestion that that prior art forms part of the common general knowledge.

### DETAILED DESCRIPTION

The present invention relates to methods of treating or preventing organophosphorus (OP) poisoning. In particular, the present invention relates to the use of intranasal formulations comprising an acetylcholinesterase inhibitor, such as rivastigmine, for treating or preventing OP poisoning. Intranasal administration may be particularly advantageous for use in treating or preventing OP poisoning in healthy adults, including those high-performance operational populations, such as military personnel and emergency first responders.

As used herein, the term “high-performance operational population” refers to a group of two or more individuals whose occupation requires the following attributes: flexibility in job assignments, a carefully selected workforce, extensive use of problem-solving teams, intensive skill training, and extensive communication between workers and management. High-performance operational populations may include, but are not limited to, military personnel and emergency first responders. Emergency first responders may include, but are not limited to, police officers, fire fighters and paramedics. High-performance operational populations, such as military personnel and emergency first responders, may be at increased risk of direct (e.g., by terrorist or military attacks) or indirect (e.g., by accidental poisoning) OP poisoning. High-performance operational populations are typically comprised of healthy adults, particularly healthy young adults.

As used herein, the term “healthy adults” refers to humans aged 18 and over with no known history of clinically significant medical disorders that could contraindicate rivastigmine administration, for example, clinically significant liver, neurological, renal, cardiovascular, respiratory (asthma), endocrinological, gastrointestinal, haematopoietic disease or neoplasm. Preferably, the healthy adults have a body mass index (BMI) between 18 and 32 (inclusive), calculated as weight (kg)/height (m²). In some embodiments, the healthy adults are healthy young adults. As used herein, the term “young adults” refers to adults aged 18-65 (inclusive), preferably aged 18-55 (inclusive).

Thus, in some embodiments, the methods of the present invention may be suitable for the treatment of young healthy adults, such as those in high-performance operational populations, e.g., military personnel and/or emergency first responders. In some embodiments, the methods of the present invention may also be suitable for the treatment or prevention of OP poisoning in the general population, including children (e.g., aged 17 and under) and adults (e.g., aged 18 and over), including elderly adults (e.g., aged 65 and over). In particular, the methods of the present invention may also be suitable for the treatment or prevention of OP poisoning in individuals susceptible to exposure, either directly or indirectly, to OP-containing pesticides, such as those living or working in or near agricultural areas, e.g., farms.

### Organophosphorus Poisoning

The methods of the present invention may be suitable for the treatment of acute or chronic OP poisoning. For example in high performance operational populations, OP poisoning is typically acute, such as during a military or terrorist attack, leading to rapid accumulation of OPCs in the CNS. However, certain individuals may experience a build-up of OPCs in the CNS over a prolonged period of time. For example, agricultural workers, or individuals living in or near agricultural areas, may experience a build-up of OPCs in the CNS due to prolonged exposure, e.g., from pesticide drift. Exposure to OPCs may occur through inhalation, skin absorption and/or ingestion. Rapid treatment can be vital to preventing CNS damage due to OP poisoning, particularly in cases of acute exposure. The current standard of treatment for OP poisoning involves removing the exposed individual from the field and administrating atropine and oximes by auto-injectors. In contrast, intranasal administration may provide a rapid, portable and effective mechanism for administration in the field.

OP poisoning may occur upon exposure to even a small amount of an OPC. Typically, OP poisoning occurs upon exposure to a topical liquid, spray, aerosol, or gaseous aerosol comprising at least one OPC, such as a nerve agent or pesticide. OP nerve agents are widely considered the deadliest form of chemical warfare agents and exposure may affect military personnel and civilians alike. Examples of OP nerve agents include, but are not limited to, sarin (2-(fluoro-methylphosphoryl)oxypropane), cyclosarin ([fluoro (methyl)phosphoryl]oxycyclohexane), soman (3 -(fluoro-methyl-pho sphoryl)oxy-2,2-dimethyl-butane), VR (N,N-diethyl-2-(methyl-(2-methylpropoxy)phosphoryl)sulfanylethanamine), VX (S-2-[diisopropylamino]O-ethyl methylphosphonothioate), tabun (ethyl N,N-dimethylphosphoramidocyanidate), Novichok agents, and combinations thereof.

Exposure to OP pesticides may also cause OP poisoning. Examples of OP pesticides include, but are not limited to, acephate, azamethiphos, azinphos ethyl, azinphos methyl, bromophos, bromophos ethyl, cadusofos, carbophenythion, chlormephos, chlorphoxim, chlorpyrifos, chlorpyrifos-methyl, chlorthiophos, chlorvinohos, coumaphos, crotoxyphos, crufomate, cyanofenphos, cyanophos, demephron -o and -s, demeton -o and -s, demeton-s-methyl, demeton-s-methylsulphon, dialifos, diazinon, dichlofenthion, dichlorvos, dicrotophos, dimefox, dimethoate, dioxabenzophos, dioxathion, disulfoton, ditalmifos, dursban, edifenphos, EPBP, EPN, ESP, ethion, ethopropos, etrimfos, famphur, fenamiphos, fenchlorphos, fenitrothion, fensulfothion, fenthion, fonofos, formothion, fosmethilan, heptenophos, isazofos, isofenphos, isothioate, isoxathion, jodfenphos, leptophos, malathion, menazon, mephosfolan, methacrifos, methamidophos, methidathion, mevinphos, monocrotophos, naled, omethoate, oxydemeton-methyl, parathion, parathion-methyl, phenthoate, phorate, phosalone, phosmet, phosphamidon, phospholan, phoxim, pirimiphos-ethyl, pirimiphos-methyl, profenofos, propaphos, propetamphos, prothiofos, prothoate, pyraclofos, pyridaphenthion, quinlphos, schradan, sulfotep, sulprofos, temephos, TEPP, terbufos, tetrachlorvinphos, thiometon, thionazin, triazophos, trichlorfon and vamidothion, or combinations thereof. In preferred embodiments, the OP pesticide is selected from parathion, fenthion, malathion, diazinon, dursban, chlorpyrifos, terbufos, acephate, phorate, methyl parathion, phosmet, azinphos-methyl, dimethoate, and combinations thereof. Other OPCs that may cause OP poisoning will be known to those skilled in the art.

The methods of the present invention may advantageously reduce or alleviate one or more of the characteristic symptoms of organophosphorus poisoning Reference to “characteristic symptoms of organophosphorus poisoning” should be understood as a reference to any one or more symptoms that may occur in an individual suffering from OP poisoning. These symptoms may be evident throughout the course of poisoning or they may be evident only transiently or periodically. Symptoms of OP poisoning may depend on the severity of poisoning. Symptoms of mild poisoning may include anorexia, headache, dizziness, weakness, anxiety, substernal discomfort, fasciculations of the tongue and eyelids, miosis and/or impairment of visual acuity. Symptoms of moderate poisoning may include nausea, salivation, bronchorrhoea, lacrimation, abdominal cramps, diarrhoea, vomiting, sweating, hypertension or hypotension and/or muscular fasciculations. CNS symptoms of OP poisoning may include the development of persistent profound neuropsychiatric, neurological deficits (epitomized by cognitive and memory impairments psychomotor performance deficits, somatic complaints, and nonspecific mental and post-traumatic stress disorder) as well as structural alterations in the human brain due to nerve cell loss (neurodegeneration). Symptoms of severe OP poisoning may include miosis or mydriasis, non-reactive pupils, dyspnoea, respiratory depression, pulmonary oedema, cyanosis, loss of sphincter control, convulsions (seizures), coma, bradycardia or tachycardia, cardiac ischaemia, cardiac dysrhythmias, hypokalaemia, and hyperglycaemia. Other symptoms of OP poisoning may include pancreatitis and muscular paralysis and/or delayed peripheral neuropathy. The immediate risk of death for patients with OP poisoning is typically related to respiratory depression and pulmonary oedema.

Initial diagnosis of OP poisoning may be based on the presence of one or more of the abovementioned characteristic symptoms. Blood and/or urine analysis may also be used to determine the level of exposure based on the presence of certain metabolites. For example, the level of inhibition of BuChE in plasma and/or AChE in red blood cells can be indicative of OP poisoning. Typically, the degree of BuChE and/or AChE inhibition is proportional to the extent of exposure. Suitable laboratory testing procedures for determining the level of inhibition of BuChE in plasma and/or AChE in red blood cells will be known to those skilled in the art. Portable testing devices are also available for use in the field, e.g., by agricultural workers, which can provide rapid results using a finger-prick blood test (e.g., the Test-mate ChE Cholinesterase Test System). Other suitable diagnostic methods and portable equipment for diagnosing OP poisoning will be apparent to those skilled in the relevant art.

### Rivastigmine

The pharmacological action of rivastigmine as a cholinesterase inhibitor is often described as a “pseudoirreversible” enzyme inhibitor, because it produces an effect that persists much longer than the drug is present in plasma (Polinsky, 1998). Like acetylcholine, rivastigmine undergoes hydrolysis by AChE. For acetylcholine, the acetyl moiety quickly dissociates from AChE (within microseconds), allowing rapid regeneration of the enzyme. For rivastigmine, the carbamyl moiety of rivastigmine remains bound for about 8.5 h in humans despite the drug having a short half-life of 1-2 h (Cutler et al., 1998; Polinsky, 1998). The metabolic pathway for the elimination of rivastigmine is integrally related to this enzyme carbamylation as it is the principal step required for cholinesterase inhibition (Cutler et al., 1998). The conversion of rivastigmine to its main phenol metabolite 3-[(1S)-1-(dimethylamino)ethyl]phenol (NAP226-90) follows Michaelis—Menten kinetics, but for all practical purposes has linear pharmacokinetics up to a 6 mg/day oral dose and at higher doses exhibits nonlinear pharmacokinetics (Cutler et al., 1998, Hossain et al., 2002). Rivastigmine exposure is likely to be primarily responsible for the clinical effect because its metabolites have very little activity against AChE, being at least 10-fold less potent than rivastigmine (Polinsky, 1998).

Rivastigmine has the following structure, and may also be provided as a pharmaceutically acceptable salt or solvate (e.g., hydrate):

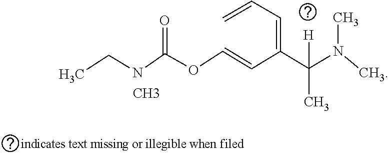

Currently, rivastigmine is available as a capsule or a solution for oral administration and as a transdermal patch. However, the orally available forms of rivastigmine are associated with significant side effects, including nausea, vomiting, diarrhoea and asthenia (Feldman and Lane 2007; Winblad et al., 2007). Additionally, oral rivastigmine has low absolute oral bioavailability of about 35% at a dose of 3 mg, and at higher oral doses of 6 mg and above it exhibits non-linear oral pharmacokinetics (Hossain et al., 2002). While the rivastigmine transdermal patch addresses some of these deficiencies, it is known to cause shown to cause skin irritation in a significant proportion of patients and may also disturb circadian rhythms resulting in disrupted sleep patterns (Lamer, 2010; Grossberg et al., 2010; Kurz et al., 2009). Consequently, the treatment discontinuation rate for the transdermal patch was found to be higher than for the oral capsule (Winblad et al., 2007).

Oral rivastigmine has previously been investigated as a pre-treatment against nerve agent poisoning in healthy young males aged 18-40 years old (Lavon et al., 2015). However, the non-linear pharmacological profile and high inter-personal variability in both drug concentration and side effects of oral rivastigmine limited its potential for use in as a pre-treatment for military personnel or first responders. While transdermal rivastigmine patches may ameliorate some of the disadvantages associated with oral administration, patches also have limited potential in high-performance applications due to their detachment risk, particularly under extreme weather conditions, and potential for skin irritation, sleep disturbances and longer time maximum rivastigmine plasma concentration (i.e., higher rivastigmine Tmax).

Most clinical studies involving rivastigmine have focused on elderly populations (e.g., aged 65 and over) and Alzheimer's patients. However, it has now surprisingly been found that intranasal administration of rivastigmine may also provide an efficacious, safe and reliable treatment for OP poisoning, particularly in healthy adults, including those in high-performance operational populations, such as military personnel and emergency first responders. In particular, intranasal administration of rivastigmine may protect the CNS from the adverse effects of OP poisoning. Further, the intranasal rivastigmine formulations used in the present invention may be well tolerated at higher doses than oral rivastigmine by young healthy adults (e.g., aged 18-55 inclusively), readily transported and administered, and may be stable under a variety of ambient conditions. The robustness of intranasal formulations disclosed herein to deviation in extreme environmental temperatures and its compact size (e.g., an exemplary nasal spray may weigh approx. 26 g and contain approx. 14 ml liquid formulation) make it particularly suitable for high-performance operational populations, for example, in existing antidotal countermeasure kits, which typically include pralidoxime/atropine auto-injectors and pyridostigmine tablets.

Advantageously, intranasal administration of rivastigmine in healthy young adults may provide increased rivastigmine exposure of oral rivastigmine but with lower exposure to the less active metabolite NAP226-90. It is estimated that the potency of NAP226-90 is 10 times less than rivastigmine (Gobburu et al., 2001). Kurz et al., (2009) indicated that oral administration of rivastigmine may result in a NAP226-90 to rivastigmine plasma ratio greater than one. Furthermore, in the case of oral administration of rivastigmine, higher plasma ratios of NAP226-90 to rivastigmine have been linked to greater and undesirable levels of peripheral anticholinesterase inhibition. As previously described, peripheral cholinesterase inhibition has been associated with increased incidence of adverse events and/or unwanted side effects, such as nausea, vomiting and diarrhoea. Without wishing to be bound by theory, presystemic metabolism due to direct exposure of rivastigmine to the gut wall or by hepatic first-pass metabolism of rivastigmine may be responsible for higher plasma ratios of the metabolite NAP226-90 to rivastigmine in the case of oral administration. Accordingly, lower plasma ratios of the metabolite NAP226-90 to rivastigmine are desirable.

In some embodiments, intranasal administration of an aqueous solution comprising rivastigmine as disclosed herein may provide a lower plasma ratio of the metabolite NAP226-90 to rivastigmine when compared with oral dosing of rivastigmine. In some embodiments, the methods according to the invention may provide a plasma ratio of NAP226-90 to rivastigmine of less than about 1.4:1, 1.2:1, more preferably less than about 1:1, still more preferably less than about 0.8:1, most preferably less than about 0.6:1.

In some embodiments, the methods of the present invention may advantageously enable rivastigmine to be administered in healthy young adults at the same or higher doses than current treatments and/or delivery methods (e.g., oral or transdermal) with a reduced incidence of side effects. For example, the intranasal rivastigmine formulations disclosed herein may be administered to healthy young adults in an amount equivalent to up to about 20 mg rivastigmine free base per day. For example, the intranasal rivastigmine formulations disclosed herein may be administered to healthy young adults in an amount equivalent to up to about 20 mg, 19 mg, 18 mg, 17 mg, 16 mg, 15 mg, 14 mg, 13 mg, 12 mg, 11 mg, 10 mg, 9 mg, 8 mg, 7 mg, 6 mg, 5 mg or 4 mg rivastigmine free base per day. Preferably, intranasal rivastigmine formulations may be administered to healthy young adults in an amount equivalent to up to about 16 mg per day, more preferably up to about 12 mg per day, even more preferably up to about 4 mg per day of rivastigmine free base.

### Rivastigmine Formulations

Intranasal rivastigmine formulations suitable for use in the present invention are described in WO/2016/049700, the entire contents of which are incorporated herein by reference. The present inventor has previously found that the use of intranasal rivastigmine formulations overcome various limitations of oral and transdermal rivastigmine formulations in treating neurological or neurodegenerative diseases or disorders, such as Alzheimer's disease and Parkinson's disease (WO/2016/049700). The advantages of intranasal administration of rivastigmine were found to include, but are not limited to, rapid absorption, fast onset of action, avoidance of hepatic first-pass metabolism and ease of administration.

It is to be understood that rivastigmine may be provided as the free base form or as a pharmaceutically salt or derivative. The term “pharmaceutically acceptable salts” includes pharmaceutically acceptable solvates (including hydrates), and pharmaceutically acceptable addition salts of rivastigmine. Pharmaceutically acceptable derivatives of rivastigmine could be provided in the form of a prodrug, which may, upon administration to a subject, be capable of providing (directly or indirectly) rivastigmine or an active metabolite or residue thereof.

In some embodiments, the pharmaceutically acceptable salts of rivastigmine may include acid addition salts, and the salts of quaternary amines and pyridiniums. For use in medicine, the salts of rivastigmine will be pharmaceutically acceptable salts, but it will be appreciated that non-pharmaceutically acceptable salts also fall within the scope of the present invention, since these are useful as intermediates in the preparation of pharmaceutically acceptable salts. A pharmaceutically acceptable salt involves the inclusion of another molecule such as an acetate ion, a succinate ion, a tartrate ion or other counter ion. The counter ion may be any organic or inorganic moiety that stabilizes the charge on the parent compound. Furthermore, a pharmaceutically acceptable salt may have more than one charged atom in its structure. When multiple charged atoms are present in the parent drug, its pharmaceutically acceptable salts will have multiple counter ions and these can be several instances of the same counter ion or different counter ions. Hence, a pharmaceutically acceptable salt can have one or more charged atoms in the parent compound and/or one or more counter ions.

Acid addition salts may be formed from rivastigmine and a pharmaceutically acceptable inorganic or organic acid including but not limited to hydrochloric, hydrobromic, sulfuric, phosphoric, methanesulfonic, toluenesulphonic, benzenesulphonic, acetic, propionic, ascorbic, citric, malonic, fumaric, maleic, lactic, salicylic, sulfamic, or tartaric acids. The counter ion of quaternary amines and pyridiniums include chloride, bromide, iodide, sulfate, phosphate, methansulfonate, citrate, acetate, malonate, fumarate, sulfamate, and tartrate. Also, basic nitrogen-containing groups may be quaternised with such agents as lower alkyl halides, such as methyl, ethyl, propyl, and butyl chlorides, bromides and iodides; dialkyl sulfates like dimethyl and diethyl sulfate; and others. The preparation of the pharmaceutically acceptable salts described above and other typical pharmaceutically acceptable salts is more fully described by Berge et al., “Pharmaceutical Salts,” J. Pharm. Sci., 1977:66:1-19.

In some embodiments, salts of the rivastigmine may be prepared from the free form of the compound in a separate synthetic step prior to incorporation into the compositions disclosed herein. In still other embodiments, salts of rivastigmine may be prepared in situ during preparation of the composition for administration. For example, the composition for administration may further comprise an appropriate acid which, upon contact with the free form of the active agent, forms a desired pharmaceutical salt in situ for administration.

In one or more embodiments, the compositions for intranasal delivery comprise rivastigmine or a pharmaceutically acceptable salt or solvate thereof Where rivastigmine is provided as a pharmaceutically acceptable salt, it may be provided as an acid addition salt. Acid addition salts of rivastigmine may include include hydrochloric, hydrobromic, sulfuric, phosphoric, methanesulfonic, toluenesulphonic, benzenesulphonic, acetic, propionic, ascorbic, citric, malonic, fumaric, maleic, lactic, salicylic, sulfamic, or tartaric acid salts. In particular, rivastigmine may be provided as a tartrate salt. In some embodiments, a rivastigmine salt may be prepared from the free form of the compound prior to incorporation into the compositions disclosed herein. In still other embodiments, the desired rivastigmine salt may be formed by addition of an appropriate acid to the free form of the compound in situ prior to administration.

In some aspects, rivastigmine could be provided in the form of a prodrug. The term “pro-drug” is used in its broadest sense and encompasses those derivatives that are converted in vivo to the active agent (i.e., rivastigmine). Such derivatives would readily occur to those skilled in the art.

Furthermore, it is recognised that rivastigmine may be in crystalline form, either as the free compound or as a solvate (e.g., hydrates) and it is intended that both forms are within the scope of the present invention. Methods of solvation are generally known within the art.

As previously described, the present invention encompasses the use of intranasal compositions comprising rivastigmine as the free base form or as a pharmaceutically salt or solvate thereof in the treatment of OP poisoning. Where specific dosages or concentrations of rivastigmine are referred to herein, it is understood that the specific dosage or concentration refers to the concentration of or equivalent to the free base of rivastigmine. Accordingly, where a pharmaceutically acceptable salt of rivastigmine is used, for example rivastigmine tartrate, a person skilled in the art would readily understand that the concentrations or dosages in respect of the salt, refers to the equivalent concentration or dosage of the free base form of rivastigmine. In some embodiments, the intranasal pharmaceutical composition is an aqueous formulation of rivastigmine or a pharmaceutically acceptable salt or solvate thereof, wherein the rivastigmine comprises about 0.05 to about 20% w/v, about 0.10 to about 15% w/v, about 0.15 to about 6% w/v, about 0.2 to about 5% w/v, about 0.3 to about 3% w/v, about 0.6 to about 2.5% w/v, about 1 to about 2% w/v; about 1.25 to about 1.75% w/v of the total formulation. In still other embodiments, the intranasal pharmaceutical composition is an aqueous formulation of rivastigmine or a pharmaceutically acceptable salt or solvate thereof, wherein the rivastigmine comprises about 0.5% to about 15% by weight of the total formulation.

The present inventor has previously shown that the combination of rivastigmine or a pharmaceutically acceptable salt or solvate thereof, a pH modifying agent and a thickening agent as described herein may advantageously balance the ease of administration by intranasal delivery with the subsequent adherence to the nasal mucosa. In particular, the aqueous formulations comprising rivastigmine, a pH modifying agent and a thickening agent described herein may offer improved balance between viscosity, sprayability, absorption, sustained release and/or enhanced bioavailability for effective intranasal administration of active agents. In particular, the specific combination of agents may offer improved trans-nasal absorption and increased residence time on the nasal mucosa to ultimately provide enhanced bioavailability and/or sustained release of the desired active agent without adversely affecting the ease of administration, in particular as an intranasal spray. Furthermore, the judicious selection of agents and components may enable low or reduced doses of an active to be administered, longer duration of action, and/or a reduced incidence of side effects when compared with other drug delivery methods. For example, the specific combination of agents of the formulations comprising rivastigmine described herein may advantageously provide a formulation that balances viscosity, sprayability and absorption such that it may be administered intranasally. Furthermore, the specific combination of agents used in these formulations, which provides a formulation that may be administered intranasally, may also advantageously provide improved methods of treating or preventing OP poisoning.

The pH modifying agent used in the intranasal compositions described herein may be any pharmaceutically acceptable pH-modifying agent which provides or adjusts the pH of the formulation to a pH in the range of about 3 to 6. Where the intranasal formulations described herein are formulated with a pH in the range of about 3 to 6, the formulation, in particular the active agent, may advantageously exhibit increased pharmaceutical stability and/or shelf life. For example, rivastigmine tartrate formulated in an aqueous intranasal spray solution with a pH range of about 3 to 6 as described herein, is pharmaceutically stable over a prolonged time period, such as at least 3 months, preferably at least 6 months, more preferably at least 1 year, even more preferably at least 2 years. Furthermore, formulating intranasal formulations with a pH in the range of about 3 to 6 as described herein may advantageously assist in solubilising the active agent in solution. Accordingly, the pH modifying agent may be any agent suitable for use and administration in an intranasal formulation which provides or adjusts the pH of the formulation to a pH in the range of about 3 to 6. In one or more embodiments, the pH modifying agent suitable for use with the invention may be a buffer. In one or more other embodiments, the pH modifying agent may be any pharmaceutically acceptable acidifying or alkalizing agent that is compatible with the other components of the compositions and which adjusts the pH of the formulation to a pH in the range of about 3 to 6. Suitable pH modifying agents for use in the invention include but are not limited to organic acids and their corresponding salts, mineral acids, alkali metal phosphates, carbonates, borates, hydroxides, base and mixtures thereof. In one or more embodiments, the pH modifying agent is selected from lactic acid, citric acid, tartaric acid, phosphoric acid, acetic acid, hydrochloric acid, nitric acid and their corresponding salts, sodium or potassium metaphosphate, sodium or potassium phosphate, sodium or potassium acetate, ammonia, sodium carbonate, sodium or potassium hydroxide, dibasic sodium phosphate, sodium borate, and mixtures thereof. In one or more other embodiments, the pH modifying agent may be a buffer. In particular, a buffer suitable for use in the invention may comprise an acid and a salt, such as the corresponding salt of the acid. Suitable buffers include, but are not limited to, citrate, phosphate, acetate and glycinate buffers, wherein the buffer adjusts or maintains the pH of the formulation to a pH in the range of about 3 to 6.

As described above, the pH modifying agent suitable for use in the present invention may be any agent which provides or adjusts the pH of the formulation to a pH in the range of about 3 to about 6, preferably in the range of about 3 to 5, more preferably about 3 to 4. In other embodiments, the pH modifying agent in accordance with the invention is an agent which provides or adjusts the pH of the formulation to a pH of about 3, about 3.4, about 3.5, about 3.6, about 3.7, about 3.8, about 4, about 4.5, about 5, about 5.5, or about 6. In one or more embodiments, the pH modifying agent is a citrate buffer. In some embodiments, the citrate buffer may comprise citric acid and a citrate salt, such as sodium citrate. In particular, the buffer may comprise from about 0.01% to about 1% by weight of the total composition. In still other embodiments, the pH modifying agent is an organic acid alone, such as citric acid.

The intranasal formulations described herein may also comprise a thickening agent. The use of a thickening agent may provide improved adherence of the formulation to the nasal mucosa without adversely affecting the ease of administration, in particular administration as an intranasal spray. Furthermore, the thickening agent may advantageously improve the trans-nasal absorption of the active agent, increase the residence time of the formulation on the nasal mucosa and/or reduce loss of the formulation via mucociliary clearance of the nasal passages. As such, the use of a thickening agent may advantageously provide enhanced bioavailability and/or sustained release of the desired active agent.

In one or more embodiments, the thickening agent suitable for use in the present invention may be any pharmaceutically acceptable, nasal mucosa-tolerant thickening agent known to those skilled in the art. The thickening agent may advantageously contribute to the controlled release of the active ingredient on the mucosal membranes. Suitable thickening agents for use in the invention include methylcellulose, ethylcellulose, hydroxy-ethylcellulose, hydroxyl propyl cellulose, hydroxy propyl methylcellulose, sodium carboxy methylcellulose, polyacrylic acid polymers, poly hydroxyethyl methylacrylate, polyethylene oxide, polyvinyl pyrrolidone, polyvinyl alcohol, tragacanth, sodium alginate, araya gum, guar gum, xanthan gum, lectin, soluble starch, gelatin, pectin and chitosan. In a preferred embodiment, the thickening agent may be polyvinyl pyrrolidone, also referred to as USP povidone or PVP.

In one or more embodiments, the intranasal formulations suitable for use in the present invention may comprise an amount of a thickening agent that improves adherence of the formulation to the nasal mucosa without adversely affecting administration of the formulation as an intranasal spray. The amount of thickening agent required to achieve a suitable balance between adherence of the formulation to the nasal mucosa and the sprayability of the formulation may vary depending on the nature of the thickening agent. The amount of a particular thickening agent required to achieve this balance can be determined by a person skilled in the art. Specifically, in one or more embodiments the thickening agent may comprise about 0.1% to about 2% by weight of the total composition, preferably the thickening agent may comprise about 0.25% to about 1.5% by weight of the total composition, more preferably the thickening agent may comprise about 0.5% to about 1% by weight of the total composition.

In one or more embodiments, the intranasal formulations suitable for use in the present invention may further comprise a sensory agent. The inclusion of a sensory agent may provide the patient with sensory feedback upon use, which allows the patient to recognize that administration has occurred, and may aid the patient's recollection of administration. For example, a sensory agent may provide direct feedback to the patient that the dose has been delivered to the correct location within the nasal passages. In particular, in some aspects the sensory agent may provide mucosal feel, pleasant aroma (smell) and/or an appealing taste. For example, when a sensory agent is delivered in an intranasal formulation, a small amount of residual formulation may be removed by mucociliary clearance of the nasal passages towards the nasopharynx and eventually swallowed to provide a pleasant taste. The inclusion of a sensory agent may also advantageously provide improved patient compliance and/or a positive psychological effect. Furthermore, in view of the fact that the sensory agent may provide direct feedback to the patient that the dose has been delivered, it advantageously eliminates the need for an ‘audible click’ within the metered-dose nasal spray device as a means of registering to the patient that a dose has been delivered. For example, a marketed fentanyl intranasal spray for the treatment of break-through pain relies on an audible click device to provide feedback to patients. Simplifying the metered-dose nasal spray device, for example by eliminating the need for an ‘audible click’, also has the benefit of reducing the cost of manufacture of a nasal spray as disclosed herein as a standard metered-dose nasal spray nozzle, actuator and bottle can be used if desired.

The inclusion of a sensory agent may also enhance the prophylactic or therapeutic effect of formulations used in the present invention. For example, the inclusion of a sensory agent may improve the delivery of rivastigmine across the nasal mucosa, assist partitioning into the mucus layer and nasal mucosa to aid absorption and/or aid in the break-up of the spray plume upon actuation of a metered-dose nasal spray nozzle, that is, by increasing and/or maintaining the desired spray plume angle upon actuation. Furthermore, the sensory agent may also adjust the viscosity in combination with the thickening agent, to further balance the ease of administration of the formulation, in particular as an intranasal spray, with the subsequent adherence of the formulation to the nasal mucosa.

In one or more embodiments, the sensory agent suitable for use with the invention may be any pharmaceutically acceptable, nasal mucosa-tolerant excipient known to those skilled in the art. In some embodiments, the sensory agent may be selected from coolants, salivating agents, and warming agents. Suitable sensory agents include, but are not limited to, a C₂ to C₄ alcohol, such as ethanol or isopropanol, menthols, terpenes, thymol, camphor, capsicum, phenol, carveol, menthol glucuronide, eucalyptus oil, benzyl alcohol, salicyl alcohol, clove bud oil, mint, spearmint, peppermint, eucalyptus, lavender, citrus, lemon, lime, hexylresorcinol, ketals, diols, and mixtures thereof Other suitable mucosa-tolerant terpenes are described in Williams and Barry (2001), incorporated herein by reference.

In some embodiments, the intranasal compositions disclosed herein may further comprise an antioxidant, surfactant, co-solvent, adhesive, stabilizer, osmolarity adjusting agent, preservative, penetration enhancer, chelating agent, sweetening agent, flavoring agent, taste masking agent, or colorant. Furthermore, some agents or components of the intranasal formulations disclosed herein may concurrently act, for example, as both a pH modifying agent and an osmolarity adjusting agent or as both sensory agent and a co-solvent. For example, where ethanol is used as a sensory agent in the formulations disclosed herein, it may further function as a penetration enhancer and/or a co-solvent. Where a given agent or component of an intranasal formulation is described herein with respect to a particular function, it is in no way taken to be limited to a single function only. It would be understood by a person skilled in the art that agents or components may additionally perform alternative or multiple functions.

Where the intranasal compositions disclosed herein comprise a co-solvent, the co-solvent may be any pharmaceutically acceptable co-solvent. Suitable co-solvents may include but are not limited to alcohols, polyvinyl alcohols, propylene glycol, polyethylene glycols and derivatives thereof, glycerol, sorbitol, polysorbates, ethanol, and mixtures thereof. In particular, the co-solvent may be selected from glycerol, propylene glycol and mixtures thereof. In still other embodiments, the co-solvent may comprise from about 1% to about 60% by volume of the total composition, preferably from about 2 to about 50%, more preferably from about 3 to about 40%, even more preferably from about 5 to about 35% by volume of the total composition. In some embodiments, the sensory agent may, for example, additionally act as a co-solvent or a penetration enhancer.

Where the intranasal compositions disclosed herein comprise a preservative, the preservative may be selected from any pharmaceutically acceptable preservative. In one or more embodiments, the preservative may be selected from benzalkonium chloride, methylparaben, ethylparaben, propylparaben, butylparaben, benzyl alcohol, sodium benzoate, phenylethyl alcohol, and benzethonium. More particularly, the preservative may include benzyl alcohol or sodium benzoate. In one or more embodiments, the preservative may comprise from about 0.1% to about 5% by weight of the total composition, preferably from about 0.2 to about 3% by weight, more preferably from about 0.3% to about 1% by weight of the total composition. In still other embodiments, the intranasal compositions disclosed herein do not contain a preservative.

In some embodiments, the intranasal compositions disclosed herein may be prepared as pharmaceutically acceptable emulsions, microemulsions, solutions, or suspensions. In particular, the compositions disclosed herein may be prepared as aqueous solutions or suspensions. Where the formulations of the present invention are aqueous solutions or suspensions, the formulations may comprise water is in an amount of greater than 50% by weight of the total composition, preferably greater than about 60% by weight of the total composition, more preferably greater than about 70% by weight of the total composition, even more preferably greater than about 80% by weight of the total composition. In still other embodiments, where the formulations disclosed herein are aqueous solutions or suspensions, water may comprise about 80% to about 99% by weight of the total composition, more preferably from about 85% to about 98% by weight of the total composition.

In addition to the active ingredients, the liquid dosage forms may comprise inert diluents commonly used in the art such as, for example, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the intranasal compositions may include adjuvants such as wetting agents, emulsifying and suspending agents, sweetening, flavoring, and perfuming agents. It is recognised that the additional inert diluents may also act as, for example, penetration enhancers, thickening agents, or co-solvents within the scope of the present invention, as previously described.

Where a carrier is used, the carrier must be pharmaceutically “acceptable” in the sense of being compatible with the other ingredients of the composition and not injurious to the subject.

General considerations in formulation and/or manufacture of pharmaceutical intranasal composition can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Pharmaceutical compositions for intranasal administration disclosed herein can be prepared by any method known in the art of pharmacology. In general, such preparatory methods include the steps of bringing rivastigmine (the “active ingredient”) into association with a carrier and/or one or more other accessory ingredients, and then, if necessary and/or desirable, shaping and/or packaging the product into a desired single- or multi-dose unit.

As used herein, a “unit dose” is discrete amount of the pharmaceutical composition comprising a predetermined amount of the active ingredient. The amount of the active ingredient is generally equal to the dosage of the active ingredient that would be administered to a subject and/or a convenient fraction of such a dosage such as, for example, one- half or one-third of such a dosage.

### Therapeutic Use

The intranasal rivastigmine formulations disclosed herein may be administered to a subject in need of such treatment, or they may be administered in a prophylactic sense. In particular, it is clear that the methods of the invention may be used prophylactically as well as for the alleviation of symptoms of OP poisoning References herein to “treatment” or the like may therefore include such prophylactic treatment, as well as therapeutic treatment of acute conditions or symptoms. Accordingly, in one or more embodiments, the present invention provides intranasal formulations for use in the therapeutic treatment of OP poisoning. In other embodiments, the present invention provides intranasal formulation for use in the prophylactic treatment of OP poisoning. Prophylactic administration of intranasal rivastigmine may be desirable, for example, in the case of military personnel, emergency first responders or agricultural workers at high risk of exposure to OPCs.

Accordingly, the present invention relates to a method of treating or preventing OP poisoning comprising administering an effective amount of an intranasal rivastigmine formulation as disclosed herein to a subject.

The present invention also relates to use of an intranasal rivastigmine formulation as disclosed herein in the preparation of a medicament for treating or preventing organophosphorus poisoning, wherein the treating or preventing comprises intranasal administration of the medicament to a subject.

The present invention further relates to a rivastigmine formulation as disclosed herein for use in treating or preventing OP poisoning in a subject.

In some embodiments, the subject in need of treatment or prevention of OP poisoning is a mammal. The term “mammal” as used herein includes humans, primates, livestock animals (e.g., horses, cattle, sheep, pigs, donkeys), laboratory test animals (e.g., mice, rats, guinea pigs), companion animals (e.g., dogs, cats) and captive wild animals (e.g., kangaroos, deer, foxes). Preferably, the mammal is a human.

The intranasal rivastigmine formulation used in the present invention is preferably a sustained-release aqueous intranasal formulation comprising rivastigmine or a pharmaceutically acceptable salt or solvate thereof, a pH modifying agent and a thickening agent, wherein the pH of the formulation is in the range of about 3 to 6. Advantageously, such formulation may provide rapid absorption and sustained, enhanced delivery of rivastigmine across nasal mucosa over a prolonged period. Preferably, the instranasal rivastigmine formulation is in the form of a nasal spray.

The terms “treat”, “treating” or “treatment” with regard to a condition refers to alleviating or abrogating the cause and/or the effects of the condition. As used herein, the terms “treat”, “treatment” and “treating” refer to the reduction or amelioration of the progression, severity and/or duration of the condition, or the amelioration of one or more symptoms (e.g., one or more discernible symptoms) of the condition (i.e., “managing” without “curing” the condition), resulting from the administration of one or more therapies (e.g., one or more therapeutic agents such as a compound or composition as disclosed herein). In specific embodiments, the terms “treat”, “treatment” and “treating” refer to the amelioration of at least one measurable physical parameter of a condition described herein. In other embodiments the terms “treat”, “treatment” and “treating” refer to the inhibition of the progression of a condition described herein, either physically by, e.g., stabilization of a discernible symptom or physiologically by, e.g., stabilization of a physical parameter, or both.

The terms “preventing” and “prophylaxis” as used herein refer to administering a medicament beforehand to avert or forestall the appearance of one or more symptoms of a condition. The person of ordinary skill in the medical art recognizes that the term “prevent” is not an absolute term. In the medical art, it is understood to refer to the prophylactic administration of a drug to substantially diminish the likelihood or seriousness of a condition, or symptom of the condition and this is the sense intended in this disclosure. As used in a standard text in the field, the Physician's Desk Reference, the terms “prevent”, “preventing” and “prevention” with regard to a condition refer to averting the cause, effects, symptoms or progression of a condition prior to the condition fully manifesting itself.

The terms “therapeutic equivalence” or “therapeutically equivalent” as used herein refer to compositions for nasal administration, which will produce the same clinical effect and safety profile and/or are pharmaceutical equivalents to other systemic treatments such as orally available compositions. For example, a therapeutic equivalent intranasal composition comprising rivastigmine may provide substantially the same efficacy and toxicity at a lower dosage strength than other systemic treatments such as orally available compositions.

In one or more embodiments, the aqueous intranasal formulations disclosed herein may advantageously provide an improved dose response, that is, for example with regard to the degree of absorption, the rate of absorption, and/or the duration of action or efficacy. Bioequivalence as used herein is understood to mean that an active agent in two or more alternative dosage forms reach the general circulation at the same relative rate and the same relative extent, that is, the plasma or serum level profile of a given active obtained by administration of the two alternative dosage forms are substantially similar. Furthermore, a person skilled in the art would recognise that the relative rate and degree of absorption of a given active may be characterised by a range of measures, including for example, the maximum plasma concentration (Cₘₐₓ), time to maximum plasma concentration (Tₘₐₓ), and/or average area under the plasma concentration versus time profile (AUC) following administration of a given dose.

In some embodiments, the aqueous intranasal formulations disclosed herein may provide a maximum therapeutic rivastigmine plasma concentration (Cₘₐₓ) of at least about 6 ng/mL. For example, the Cₘₐₓ may be at least about 6 ng/mL, 7 ng/mL, 8 ng/mL, 9 ng/mL, 10 ng/mL, 11 ng/mL, 12 ng/mL, 13 ng/mL or 14 ng/mL. Preferably, when the aqueous intranasal formulations of the invention are administered to healthy young adults, the Cₘₐₓ may be at least about 7.5 ng/mL, more preferably at least about 10 ng/mL, even more preferably at least about 14 ng/mL. Additionally, in one or more further embodiments, the time to maximum rivastigmine plasma concentration (Tₘₐₓ) is less than 3 hours, more preferably less than 2 hours, even more preferably less than 1.5 hours, even more preferably less than 1 hour following administration of an initial dose at time equals zero hours. Furthermore, in one or more embodiments, the aqueous intranasal formulations disclosed herein may provide an average plasma rivastigmine AUC greater than 10 ng.h per ml per mg dose of rivastigmine.

The intranasal formulations disclosed herein are to be administered to the person in need thereof in a treatment effective amount. In some embodiments, a treatment effective amount is a therapeutically effective amount or a prophylactically effective amount. The term “therapeutically effective amount” as used herein means an amount of rivastigmine sufficient to treat or alleviate the symptoms associated with OP poisoning. The therapeutically effective amount of the compound to be administered will be governed by such considerations, and is either, an incremental maximum tolerated dose, or the minimum amount, necessary to ameliorate, cure, or treat the condition or one or more of its symptoms. The term “prophylactically effective amount” refers to an amount effective in preventing or substantially lessening the chances of acquiring a disease or disorder or in reducing the severity of the disease or disorder before it is acquired or reducing the severity of one or more of its symptoms before the symptoms develop. Roughly, prophylactic measures are divided between primary prophylaxis (to prevent the development of a disease or symptom) and secondary prophylaxis (whereby the disease or symptom has already developed and the patient is protected against worsening of this process).

As used herein, the term “effective amount” relates to an amount of rivastigmine which, when administered according to a desired dosing regimen, provides the desired therapeutic activity. Suitable dosages lie within the range of about 0.1 ng per kg of body weight to 100 g per kg of body weight per dosage. The dosage may be in the range of 1 μg to 10 g per kg of body weight per dosage, such as is in the range of 1 mg to 1000 mg per kg of body weight per dosage. In one embodiment, the dosage may be in the range of 1 mg to 500 mg per kg of body weight per dosage. In another embodiment, the dosage may be in the range of 1 mg to 250 mg per kg of body weight per dosage. In yet another embodiment, the dosage may be in the range of 1 mg to 200 mg per kg of body weight per dosage, such as up to 50 mg per kg body weight per dosage.

In some embodiments, a single dose may be sufficient to treat or prevent OP poisoning. A single dose may be delivered in one or more aliquots (e.g., one or more sprays per nostril) to achieve the desired dose. In other embodiments, multiple doses may be required to treat or prevent OP poisoning. Dosing may occur at intervals of minutes, hours, days, weeks, months or years or continuously over any one of these periods. In particular, the treatment of OP poisoning may require shorter dosage intervals than the treatment of other indications (e.g., Alzheimer's disease), such as 24 hours, 12 hours, 8 hours, 6 hours, 4 hours, 3 hours, 2 hours, 1 hour, 30 minutes, 15 minutes, 10 minutes, 5 minutes, or less. The administered amount may be an amount sufficient to treat or alleviate the symptoms associated with OP poisoning. Where administration of rivastigmine is associated with one or more side effects, dosing may occur at intervals sufficient to treat or alleviate the symptoms of OP poisoning and concurrently minimise or reduce the associated side effects. In one or more embodiments, intranasal formulations disclosed herein may provide adjustable, individualised dosing, which may advantageously minimise undesirable cholinergic burden whilst delivering an effective dose for the treatment of OP poisoning

The terms “administer”, “administering” or “administration” in reference to a compound, composition or formulation disclosed herein means introducing the active agent (i.e., rivastigmine) into the system of the subject in need of treatment. When the active agent is provided in combination with one or more other active agents, “administration” and its variants are each understood to include concurrent and/or sequential introduction of rivastigmine and the other active agents.

In certain embodiments, an effective amount of an active agent for administration one or more times a day to a 70 kg adult human may comprise about 0.0001 mg to about 4000 mg, about 0.0001 mg to about 3000 mg, about 0.0001 mg to about 200 mg, about 0.001 mg to about 1500 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of an extract or compound per unit dosage form.

In certain embodiments, the intranasal compositions disclosed herein may be at dosage levels sufficient to deliver from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, and from about 1 mg/kg to about 25 mg/kg, of subject body weight per day, one or more times a day, to obtain the desired therapeutic effect.

The intranasal formulations disclosed herein may be administered in a single dose or a series of doses. Suitable dosage amounts and dosing regimens can be determined by the attending physician or a trained non-physician in the field and may depend on the severity of OP poisoning or associated symptoms as well as the general age, health and weight of the subject. It will be appreciated that dose ranges as described herein provide guidance for the administration of provided pharmaceutical formulations to an adult. The amount to be administered can be determined by a medical practitioner, appropriately trained non-physician or person skilled in the art. In some embodiments, a physician will not be present to administer an intranasal rivastigmine formulation as disclosed herein, particular in the case of acute OP poisoning. In such situations, the dosing regimen may be decided by a non-physician based on the presence and/or severity of one or more characteristic symptoms of OP poising and/or by using a suitable field test.

The formulations disclosed herein may be administered to a person in need thereof by any suitable intranasal delivery methods. Suitable methods for intranasal administration would be well known to a person skilled in the art. The intranasal formulations disclosed herein can be administered as a spray or drop. Accordingly, suitable commercial packages containing the intranasal formulation can be in any spray container known in the art. In one or more embodiments, the formulations disclosed herein may be administered via a spray device or container. Spray devices may be single unit dose systems or multiple dose systems, for example comprising a bottle, a pump and/or an actuator. Such spray devices are available commercially. Suitable commercial spray devices include those available from Nemera, Aptar, Bespak and Becton-Dickinson. In still other embodiments, the formulations disclosed herein may be administered via an electrostatic spray device, such as described in U.S. Pat. No. 5,655,517. Other suitable means for administering formulations intranasally in accordance with the invention include via a dropper, a syringe, a squeeze bottle, and any other means known in the art for applying liquids to the nasal mucosa in an accurate and repeatable fashion.

The spray devices used to administer the formulation can range from single-use metered-dose spray devices, multiple-use metered dose nasal spray devices and are not limited to spraying the solutions into each naris but can be administered as a gentle liquid stream from a plunger, syringe or the like or as drops from a unit-dose or multi-dose squeeze bottle, or other means known in the art for applying liquids to the nasal mucosa in an accurate and repeatable fashion.

In one or more embodiments, a spray device suitable for use with the invention may typically deliver a volume of liquid in a single spray actuation in the range of from 0.01 to 0.15 mL. A typical dosing regimen for a nasal spray product may be in the range of one spray into a single nostril (naris) to two sprays into each nostril (naris). Repeat dosing of the same nostril (naris) may also be undertaken. It is recognised that the dosing schedule, including a repeat dosing schedule, may be modified to obtain a desired pharmacokinetic profile. Further, the dosing schedule may be modified to achieve a rapid reduction in severity, preferably cessation, of symptoms of OP poisoning, such as pulmonary secretions and bronchoconstriction. In one or more embodiments, repeat dosing may occur every 1 min, 2 min, 3 min, 4 min, 5 min, 6 min, 7 min, 8 min, 9 min, 10 min, 15 min, 20 min, 30 min, 45 min, 1 hour, or more. In particular, incremental increases in repeat dosing may be required to achieve a reduction in severity or cessation of symptoms of OP poisoning. For example, it may be necessary to increase each repeat dose by 25%, 50%, 75%, 100%, 150% or 200% in order to achieve a reduction in severity or cessation of symptoms of OP poisoning.

The amount of rivastigmine administered per dose or the total volume of formulation administered will depend on such factors as the nature and severity of the symptoms, the age, weight, and general health of the patient. In still other embodiments, repeat dosing may occur where a patient does not adequately respond to an initial dose, for example, by alleviation of one or more symptoms of OP poisoning. In some embodiments, the intranasal pharmaceutical composition may deliver a unit dose of rivastigmine selected from about 0.05 to about 20 mg, about 0.10 to about 15 mg, about 0.15 to about 12 mg, about 0.2 to about 8 mg, about 0.4 to about 4 mg, about 0.6 to about 2 mg, about 1 to about 4 mg or about 4 to about 16 mg.

It is recognised that relative amounts of excipients, solvents, diluents, salts, thickening agents, sensory agents, buffers, and/or any additional ingredients in a pharmaceutical composition as disclosed herein may vary, depending upon the identity, size, and/or condition of the subject treated.

In certain embodiments, unit dosage compositions are those containing a daily dose or unit, daily sub-dose, as herein above described, or an appropriate fraction thereof, of the active ingredient.

In certain embodiments, it is envisaged that the intranasal formulations comprising rivastigmine described herein may be administered to a person in need thereof as a substitute or replacement for other traditional medication. In other embodiments, it is envisaged that intranasal formulations comprising rivastigmine as disclosed herein be administered to a subject in need thereof as a supplement or adjunct to traditional medication. In still other embodiments, it is envisaged that intranasal formulations comprising rivastigmine in accordance with the invention may be administered to a subject in need thereof in the absence of adjunct therapy.

Preferably, the methods of treating OP poisoning according to the present invention are to be used in conjunction with standard manual decontamination processes for patients known to have or suspected of having OP poisoning. For example, the subject should be removed from the field (where necessary), the subject's clothing should be removed and destroyed and their skin flushed with water. Dry agents such as flour, sand or bentonite may also be applied to the skin of the subject to absorb residual OPCs. The methods of treating OP poisoning according to the present invention also do not alleviate the need for any persons treating subjects known to have or suspected of having OP poisoning to wear personal protective equipment to avoid contamination.

Replacing traditional medication for the treatment of OP poisoning with an intranasal formulation comprising rivastigmine disclosed herein may be advantageous, particularly where the traditional medication is associated with one or more adverse effects (for example, nausea, vomiting, diarrhoea). One of skill in the art will also be familiar with the difficulties in administering traditional medications, including lag phases before the effects are observed, and/or systemic dosage concentration peaks and troughs following administration. Examples of traditional medications would be known to those skilled in the art and include, but are not limited to, atropine, pralidoxime, a benzodiazepine (e.g., diazepam), pyridostigmine and galantamine.

While human experiments involving nerve agents such as OPCs are considered unethical, an example of an animal model suitable for testing the prevention of organophosphorus poisoning is described in Lane et al. (2020). This animal study was conducted in Cynomolgus monkeys who received pre-treatment with oral galantamine to estimate its potential for preventing soman poisoning in humans. Cynomolgus monkeys pre-treated with 1.5 mg/kg or 3.0 mg/kg oral galantamine (and post-treated with conventional antidotes, i.e., atropine, 2-PAM, and midazolam) showed delayed onset and/or reduced severity of clinical signs of soman toxicity, However, following the methods and dosing regimen described by Lane et al. (2020), a 70 kg healthy adult human would require oral pre-treatment with 105 mg to 210 mg in order to reduce the severity of soman poisoning. Meanwhile, it has been shown that Alzheimer's patients given either 24 or 32 mg per day of galantamine (Wilcock et al. 2000) or 16 mg per day of galantamine (Tariot et al. 2000) cause adverse side effects, such as nausea, vomiting and diarrhea. Accordingly, oral administration of about 105 mg to 210 mg galantamine to a healthy adult would be expected to cause even higher rates and/or severity of adverse side effects, which is particularly undesirable in high performance operational populations, such as military personnel and emergency first responders.

In contrast, the present inventor has found that intranasal administration of a rivastigmine formulation as disclosed herein may advantageously reduce the side effects that occur with existing pre-treatments, including galantamine oral pre-treatment. Further, the intranasal rivastigmine formulations as disclosed herein may be more effective than existing pre- and/or post-treatments at inhibiting both acetylcholinesterase and butyrylcholinesterase enzymes in the brain so as to protect the central nervous system from neuronal damage and its associated cognitive, functional and behavioural decline.

### Combination Therapy

In other embodiments, the intranasal formulations comprising rivastigmine disclosed herein may be administered to a subject in need thereof, together with other medication for a discrete period of time, to address specific symptoms. In still other embodiments, the person in need thereof may be treated with both an intranasal formulation comprising rivastigmine and one or more additional medications (administered sequentially or in combination) for the duration of the treatment period. Such combination therapy may be particularly useful, for example, where an additive or synergistic therapeutic effect is desired.

The intranasal formulations disclosed herein may be used in combination therapy with one or more additional therapeutic agents. For combination treatment with more than one active agent, where the active agents are in separate dosage formulations, the active agents may be administered separately or in conjunction. In addition, the administration of one element may be prior to, concurrent to, or subsequent to the administration of the other agent.

The phrase “combination therapy” as used herein, is to be understood to refer to administration of an effective amount, using a first amount of for example rivastigmine or a pharmaceutically acceptable salt or solvate thereof as described herein, and a second amount of an additional suitable therapeutic agent.

When co-administered with other agent, an “effective amount” of the second agent will depend on the type of drug used. Suitable dosages are known for approved agents and can be adjusted by a person skilled in the art according to the condition of the subject, the type of condition(s) being treated and the amount of a compound, extract or composition being used. In cases where no amount is expressly noted, an effective amount should be assumed. For example, compounds described herein can be administered to a subject in a dosage range from between about 0.01 to about 10,000 mg/kg body weight/day, about 0.01 to about 5000 mg/kg body weight/day, about 0.01 to about 3000 mg/kg body weight/day, about 0.01 to about 1000 mg/kg body weight/day, about 0.01 to about 500 mg/kg body weight/day, about 0.01 to about 300 mg/kg body weight/day, about 0.01 to about 100 mg/kg body weight/day.

In certain embodiments, rivastigmine or a pharmaceutically acceptable salt or solvate thereof, and the additional therapeutic agent, are each administered in an effective amount (i.e., each in an amount that would be therapeutically effective if administered alone). In other embodiments, rivastigmine or a pharmaceutically acceptable salt or solvate thereof, and the additional therapeutic agent are each administered in an amount that alone does not provide a therapeutic effect (a sub-therapeutic dose), but together provide a therapeutic effect. In yet other embodiments, rivastigmine or a pharmaceutically acceptable salt or solvate thereof, can be administered in an effective amount, while the additional therapeutic agent is administered in a sub-therapeutic dose. In still other embodiments, rivastigmine or a pharmaceutically acceptable salt or solvate thereof, can be administered in a sub-therapeutic dose, while the additional therapeutic agent is administered in an effective amount.

As used herein, the terms “in combination” or “co-administration” can be used interchangeably to refer to the use of more than one therapy (e.g., one or more prophylactic and/or therapeutic agents). The use of the terms does not restrict the order in which therapies (e.g., prophylactic and/or therapeutic agents) are administered to a person in need thereof

Co-administration encompasses administration of the first and second amounts of therapeutic compounds in an essentially simultaneous manner, such as in a single pharmaceutical composition, for example, an intranasal spray having a fixed ratio of first and second amounts. In addition, such co-administration also encompasses use of each compound in a sequential manner in either order. When co-administration involves the separate administration of the first amount of rivastigmine or a pharmaceutically acceptable salt or solvate thereof, and a second amount of an additional therapeutic agent, they are administered sufficiently close in time to have the desired therapeutic effect. For example, the period of time between each administration which can result in the desired therapeutic effect, can range from minutes to hours and can be determined taking into account the properties of each compound such as potency, solubility, bioavailability, plasma half-life, and kinetic profile. For example, rivastigmine or a pharmaceutically acceptable salt or solvate thereof, and the second therapeutic agent can be administered in any order within about 24 hours of each other, within about 16 hours of each other, within about 8 hours of each other, within about 4 hours of each other, within about 1 hour of each other, within about 30 minutes of each other, within about 15 minutes of each other, within about 10 minutes of each other or within about 5 minutes of each other. In the event of acute OP poisoning, it may be advantageous to administer rivastigmine or a pharmaceutically acceptable salt or solvate thereof, and the second therapeutic agent within about 5 minutes of each other, e.g., within about 5 min, 4 min, 3 min, 2 min, 1 min, 30 seconds or less of each other.

In one or more embodiments where the rivastigmine or a pharmaceutically acceptable salt or solvate thereof is administered with an additional therapeutic agent, the additional therapeutic agent may be any therapeutic agent that provides a desired treatment outcome. In particular, the additional therapeutic agent may be selected from known therapeutic agents for the treatment or prevention of OP poisoning including, but not limited to, atropine, pralidoxime, a benzodiazepine (e.g., diazepam), pyridostigmine, galantamine, and combinations thereof

Atropine (an enantiomeric mixture of d-hyoscyamine and 1-hyoscyamin) is currently recognised as the first line of treatment of OP poisoning. Atropine is a muscarinic antagonist, which competes with acetylcholine for binding at muscarinic receptors, thereby reducing the adverse effects associated with excessive acetylcholine during OP poisoning. Relatively large doses of atropine are required to treat OP poisoning compared to other indications. Typically, the dosage of atropine is doubled every 3 to 5 min until excessive pulmonary secretions are reduced and bronchoconstriction ceases. A typical initial dose for adults is 2 to 5 mg IV or 0.05 mg/kg IV for children until the adult dose is reached. Severe OP poisoning may require several hundreds of milligrams of atropine before symptoms improve. However, atropine may be associated with side effects including ventricular fibrillation, supraventricular or ventricular tachycardia, dizziness, nausea, blurred vision, loss of balance, dilated pupils, photophobia, dry mouth and potentially extreme confusion, hallucinations, and excitation, particularly in older adults. Further, atropine is typically provided as the sulfate salt, which can cause histamine release and anaphylaxis to susceptible due to the ability of atropine to cross the BBB.

As atropine only acts on muscarinic receptors, it is typically co-administered with pralidoxime (and, if available, oxygen). However, pralidoxime should be administered subsequently to atropine to avoid worsening of muscarinic-mediated symptoms (e.g., salivation, lacrimation, urination, diarrhea, GI upset, emesis, hypotension, diaphoresis, miosis, bradycardia, bronchospasm and bronchorrhea). Pralidoxime (2-PAM; 1-methylpyridine-6-carbaldehyde oxime) is a member of the oxime family of compounds, which works by binding to AChE that have been inactivated by OPCs. Certain OPCs (e.g., sarin) bind to serine hydroxyl group located at the active site of AChE, inhibiting its ability to break down ACh. 2-PAM is able to bind to a different portion of the active site of AChE (the anionic site), where it displaces the phosphate moiety of the OPC from the serine residue to form a phosphorylated oxime. The resulting phosphorylated oxime unbinds from the active site to regenerate the functional AChE. A typical dose of pralidoxime involves IV administration of at least 30 mg/kg (approx. 2-5 mg) to adults or 10-20 mg/kg for children over 15-30 min to avoid respiratory or cardiac arrest. Administration may be required every 60 min for several days depending on the severity of symptoms. In addition to the risk of respiratory or cardiac arrest if administered too rapidly, 2-PAM can also cause dizziness, blurred vision, diplopia and impaired accommodation, headache, drowsiness, nausea, tachycardia, hyperventilation, maculopapular rash and muscular weakness, although these symptoms are typically masked by the symptoms of OP poisoning.

Benzodiazepines, particularly diazepam, are typically used as an adjunct to other treatments for OP poisoning (e.g., atropine/pralidoxime) to treat or prevent seizures. Diazepam may also ameliorate muscle fasciculations associated with OP poisoning. Preferably, diazepam is used in a prophylactic sense to prevent seizures and/or muscle fasciculation. Diazepam is typically administered intramuscularly in the field via auto-injector together with atropine and pralidoxime. However, intramuscular absorption is poor and, where possible, diazepam should be administered intravenously. A typical dose of diazepam for the treatment of OP poisoning may range from 2.5 mg to 10 mg. While diazepam is associated with serious side effects, such as changes mental/mood (such as memory problems, agitation, hallucinations, confusion, restlessness, depression), trouble speaking, trouble walking, muscle weakness, shaking (tremors), trouble urinating, yellowing eyes/skin and signs of infection, its ability to protect the CNS from long term damage is generally considered to outweigh the risk of these side effects.

Pyridostigmine is a quaternary carbamate compound used as prophylactic treatment for OP poisoning by inhibiting AChE, slowing the hydrolysis of acetylcholine. It is typically administered in the form a bromide salt. Pyridostigmine does not readily penetrate the blood brain barrier (BBB) and does not protect from OP-induced major CNS symptoms. Accordingly, pyridostigmine administration is typically followed by an antidotal mixture of atropine and pralidoxime. A typical dose of pyridostigmine is 30 mg orally, preferably administered several hours before OP exposure. If symptoms of OP poisoning appear, treatment with pyridostigmine is typically replaced with atropine/pralidoxime. Adverse effects of pyridostigmine include sweating, diarrhea, nausea, vomiting, abdominal cramps, increased salivation, tearing, increased bronchial secretions, constricted pupils, vasodilation and erectile dysfunction.

Galantamine is a cholinesterase inhibitor, typically used to treat dementia associated with Alzheimer's disease. However, it has been identified as a potential treatment for OP poisoning based on testing in guinea pigs exposed to lethal doses of soman (Aracava et al., 2009). As previously discussed, galantamine also has the disadvantage of poor tolerability in humans after oral administration (Wilcock et al., 2000).

Accordingly, in some embodiments, an intranasal rivastigmine formulation as disclosed herein may be co-administered with an additional therapeutic agent selected from atropine, pralidoxime, a benzodiazepine, pyridostigmine, galantamine, and combinations thereof The additional therapeutic agent may be administered sequentially or in combination with an intranasal rivastigmine formulation as disclosed herein.

Where rivastigmine is administered in combination with an additional therapeutic agent, the second agent may be administered in any “effective amount” which provides the desired therapeutic activity, as described above. Suitable dosage amounts and dosing regimens of the additional therapeutic agent can be determined by the attending physician (or non-physician) and may depend on the severity of OP poisoning or a symptom thereof, as well as the general age, health and weight of the subject. It will be appreciated that, unless otherwise specified, dose ranges as described herein provide guidance for the administration of provided pharmaceutical compositions to an adult human. The amount to be administered to can be determined by a medical practitioner or person skilled in the art.

### Kits

The intranasal formulations disclosed herein may be contained in a kit. The kit may include, for example, rivastigmine and an additional agent, each packaged or formulated individually for intranasal administration, or packaged or formulated in combination. Thus, rivastigmine may be present in a first container, and the kit can optionally include one or more agents in a second container. The container or containers may be placed within a package, and the package can optionally include administration or dosage instructions. A kit can include additional components such as syringes or other means for administering the agents as well as diluents or other means for formulation. Thus, the kits can comprise: a) a pharmaceutical composition comprising rivastigmine described herein and a pharmaceutically acceptable carrier, vehicle or diluent; and b) a container or packaging. The kits may optionally comprise instructions describing a method of using the pharmaceutical compositions in one or more of the methods described herein (e.g., preventing or treating one or more of the diseases and disorders described herein). The kit may optionally comprise a second pharmaceutical composition comprising one or more additional agents described herein for co-therapy use, a pharmaceutically acceptable carrier, vehicle or diluent. The pharmaceutical composition comprising rivastigmine and the second pharmaceutical composition contained in the kit may be optionally combined in the same pharmaceutical composition.

Additionally or alternatively, the intranasal formulations disclosed herein may be contained in a countermeasure kit, preferably in the form of a nasal spray, together with one or more additional therapeutic agents for the treatment of OP poisoning. For example, the countermeasure kit may comprise an intranasal rivastigmine formulation as disclosed herein and one or more additional therapeutic agents including, but not limited to atropine, pralidoxime, a benzodiazepine (e.g., diazepam), pyridostigmine and galantamine. In some embodiments, the countermeasure kit may comprise an auto-injector for administration of one or more additional therapeutic agents. In some embodiments, the countermeasure kit may comprise a diagnostic test for OP poisoning. The diagnostic test may be any suitable field test known in the art, for example, a finger-prick test (e.g., the Test-mate ChE Cholinesterase Test System). Preferably, a countermeasure kit for use according to the present invention should be lightweight and portable for ease of use in the field.

It may also be desirable to provide a written memory aid in the kit containing information and/or instructions for the physician, pharmacist or subject regarding when the medication is to be taken. An example of such a memory-aid is a mechanical counter that indicates the number of daily doses that have been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

Those skilled in the art will be aware that the invention described herein is subject to variations and modifications other than those specifically described. It is to be understood that the invention described herein includes all such variations and modifications. The invention also includes all such steps, features, methods, compositions and compounds referred to or indicated in this specification, individually or collectively, and any and all combinations of any two or more of said steps or features.

Certain embodiments of the invention will now be described with reference to the following examples which are intended for the purpose of illustration only and are not intended to limit the scope of the generality hereinbefore described.

### EXAMPLES

### Example 1

### Pharmacokinetic and Safety Study of Rivastigmine Intranasal Spray and Exelon® Oral Capsule in Healthy Young Adults

A randomised, crossover, two period, pharmacokinetic, bioavailability and safety study of a single dose of a rivastigmine nasal spray according to the present invention and single dose rivastigmine (Exelon®, Novartis) oral capsule was conducted in young healthy adult males aged 18-55 (n=16).

### Methods

The study was approved by The Alfred Hospital Human Research and Ethics Committee, Melbourne, Australia and prospectively registered with the Australia and New Zealand Clinical Trial Registry (ANZCTR), Trial ID ACTRN12619001513101. The study was conducted at the clinical trial unit of Nucleus Network Limited (Melbourne, Australia) and independently monitored by Avance Clinical Pty Ltd (Adelaide, Australia) in accordance with Good Clinical Practice (GCP) and the principles of the Declaration of Helsinki.

### Design

The study was of a randomised, crossover, two period design in 16 young healthy adult males volunteers who all gave written informed consent to participate in the study.
Treatment A: Rivastigmine nasal spray (4 mg) administered as a single dose (one spray each nostril).
Treatment B: Rivastigmine oral capsule (Exelon®; 3mg) as a single dose.

Participants were randomised to receive each treatment as a crossover with a single dose given on the first day, followed by a 2-day washout and a single dose given on the fourth day.

Key inclusion criteria for participation in the study included:
Healthy males between 18 and 55 years (inclusive) of age.
No known history of clinically significant neurological, renal, cardiovascular, respiratory (asthma), endocrinological, gastrointestinal, haematopoietic disease, neoplasm or any other clinically significant medical disorder, which in the Principal Investigator's judgment contraindicate administration of the study interventions.
BMI 18-32 (inclusive) calculated as Weight (kg)/Height (m²).

Key exclusion criteria included:
Known hypersensitivity to rivastigmine, components (benzyl alcohol, benzoates), other carbamates or ondansetron.
Current symptomatic allergic rhinitis.
Presence of significant disease or anatomical abnormality affecting the nasal passages.
History of or currently active asthma or chronic obstructive pulmonary disease, excluding childhood asthma.
Use of any prescription, OTC or herbal medication within 7 days or 5 half-lives (whichever is longer) of study drug administration, with the exception of the oral contraceptive pill, paracetamol up to 2 g daily and low-moderate dose NSAID.
History of or currently active cardiac arrhythmias such as bradycardia and sick sinus syndrome.
History of heart block or disease of cardiac conducting system.
History of urinary tract obstruction.
History of or currently active GI diseases such as peptic ulcer, GERD, bleeding or history of any GI surgery other than appendectomy or herniotomy, or with any gastrointestinal disorder likely to influence drug absorption, or with any history of anorexia, frequent nausea or emesis, regardless of aetiology.

### Clinical

Detailed in Table 1 is the intranasal spray formulation in accordance with the invention administered during the clinical trial:

TABLE 1

| Component | % composition | |
|---|---|---|
| Rivastigmine tartrate | 3.2% | w/v |
| Citric acid monohydrate | 0.055% | w/v |
| Carveol | 0.05% | v/v |
| Ethanol | 10% | v/v |
| Benzyl alcohol | 0.65% | v/v |
| Polyvinyl pyrrolidone | 1% | w/v |

| | |
|---|---|
| Distilled water | to volume 100 mL |

The abovementioned formulation was filled into 15 ml high-density polyethylene bottles and sealed with 100 μL Advancia metered-dose pump valves with nasal spray actuators and caps (Nemera, Le Treport, FR) and had a final pH of 3.6. One 100 μL spray (3.2 mg rivastigmine tartrate equivalent to 2 mg rivastigmine free base) was administered into each nostril by study staff for a total single-dose of 4 mg rivastigmine. The shot weight delivered to each participant was determined by weighing the intranasal spray device before and after each dosing each participant.

The Exelon® oral capsule formulation administered during the clinical trial contained 4.8 mg rivastigmine tartrate equivalent to 3 mg rivastigmine free base.

Blood samples were taken from the non-dominant arm of each participant for the intranasal treatment at time point 0 (pre-administration), 5 min, 10 min, 15 min, 30 min, 45 min 60 min, 90 min, 2 h, 3 h, 4 h, 6 h, 8 h, 10 h, 12 h and 24 h post-administration. Blood samples were taken from the non-dominant arm of each participant for the oral treatment at time point 0 (pre-administration), 15 min, 30 min, 45 min, 60 min, 90 min, 2 h, 3 h, 4 h, 6 h, 8 h, 10 h, 12 h, 15 h and 24 h.

Blood was collected into pre-chilled 1(2 EDTA tubes and centrifuged at 3000 rpm (about 1900 g), 3° to 5° C., for 10 min, and the harvested plasma immediately transferred to a pre-chilled polypropylene centrifuge tube, containing sodium fluoride (110 μL of a 1M sodium fluoride solution per 1 mL plasma) to inhibit any ex vivo enzymatic breakdown of the parent compound and its metabolite and stored frozen at −20° C. pending analysis.

Visual nasal mucosal examination occurred at screening, check-in, pre-nasal-dose and 24 hours postnasal-dose. Adverse events were monitored from Day 1 until Day 5 (24 hours post-dose) and at follow-up visit (Day 9 +/−1).

Healthy screens were undertaken at screening, admission (Day-1) and on Day 5 (24 hours post-dose). A healthy screen pre- and post-study was completed for all participants. The healthy screen included:
Physical examination, including vital signs (blood pressure, heart rate, respiratory rate and temperature) and ECG;
Urinalysis for general health (urine pH, blood, protein, ketones, leukocyte elastase, nitrates, glucose, Specific gravity, urobilinogen and bilirubin) and any drugs of addiction*. Blood collected for clinical laboratory analysis (serum chemistry and haematology) and analysis for HIV*, hepatitis B*, and hepatitis C* analysis. Normal ranges for clinical laboratory parameters will be as per the local laboratory definitions (*pre-study).

### Bioanalytical Assay

Analysis of rivastigmine and its primary metabolite, NAP226-90 was performed by the bioanalytical division of Anapharm Bioanalytics (Barcelona, SP) using a validated LC/MS/MS bioanalytical assay. Rivastigmine, NAP226-90 and internal standards were extracted from an aliquot of human EDTA plasma using a liquid-liquid extraction procedure with ethyl acetate and then injected into a liquid chromatograph equipped with a tandem mass spectrometry detector (LC/MS/MS). The calibration range used for this assay was from 0.05 to 15 ng/mL for rivastigmine and from 0.05 to 7.5 ng/mL for NAP226-90. The assay passed linearity for rivastigmine and NAP226-90 over each of the calibration ranges tested.

### Non-Compartmental Pharmacokinetic Analysis

Area under the curve (AUC) was taken from the blood plasma concentration-time profile. AUC to the last measured concentration (AUCO-1) and AUC zero to infinity (AUCO-∞) were calculated by the linear trapezoidal rule using PKPlus (version 2.5; Simulations Plus, Lancaster, Calif., USA). The maximum plasma concentration (Cₘₐₓ) and time to maximum plasma concentration (Tₘₐₓ) were determined by the software and checked by visual inspection of the data. Terminal elimination half-life (t_{1/2}) was defined as 0.693/λ, where is the terminal elimination rate constant (calculated from the slope of the regression line of the terminal phase of the natural logarithm of concentration versus time). Fluctuation index (FI) equalled (Cₘₐₓ) divided by C_{avg0-1}. C_{avg0-1} was calculated as AUCO-1 divided by the time for the last measured concentration. Metabolite (NAP226-90) to parent ratio was calculated by dividing AUCO-∞ of NAP-226-90 divided by AUCO-∞ of rivastigmine.

Table 3 details the non-compartmental pharmacokinetic parameters of rivastigmine (RIV) and NAP226-90 after administration of 3 mg as an oral capsule (Exelon®) in healthy young adult men (n=16).

TABLE 3

| | | | AUC | | | |
|---|---|---|---|---|---|---|
| | | | (0, ∞)_{OR} | | | |
| | C_{max, OR} | t_{max, OR} | (ng · h · | t_{1/2, OR} | | |
| RIV | (ng · ml⁻¹) | (h) | ml⁻¹) | (h) | | FI, _{OR} |
| Mean | 8.4 [6.0] | 1.2 | 19.6 [13.2] | 1.1^{a} | | 3.8 |
| Min-max | 1.1-26.8 | 0.5-2.0 | 1.7-51.0 | 0.8-2.2 | | 2.4-6.3 |
| CV (%) | 81 | 40 | 76 | | | 31 |
| 25^{th}, 75^{th}† | | | | 0.9, 1.4 | | |
| | | | AUC | | | |
| | | | (0, ∞)_{OR} | | | |
| NAP226- | C_{max, OR} | t_{max, OR} | (ng · h · | t_{1/2, OR} | | |
| 90 | (ng · ml⁻¹) | (h) | ml⁻¹) | (h) | N:R, _{OR} | FI, _{OR} |
| Mean | 3.9 [3.8] | 1.8 | 22.9 [22.4] | 3.1 | 1.38^{a} | 3.7 |
| Min-max | 2.5-6.5 | 0.5-4.0 | 16.1-34.6 | 2.3-4.2 | 0.55-9.42 | 2.3-5.5 |
| CV (%) | 27 | 51 | 23 | 18 | | 26 |
| 25^{th}, 75^{th}† | | | | | 0.85, 2.95 | |

Cmax, peak plasma concentration;
tmax, time of Cmax;
AUC(0, ∞), area under the plasma concentration-time curve from time zero to infinity;
t_{1/2}, terminal half-life;
FI, Fluctuation Index;
N:R, NAP226-90 AUC(0, ∞) to rivastigmine AUC(0, ∞) ratio;
NS, nasal;
^{a}median;
†25^{th} and 75^{th} percentiles;
[geometric mean].

Table 4 details the non-compartmental pharmacokinetic parameters of rivastigmine (RIV) and NAP226-90 after administration of 4 mg rivastigmine nasal spray (one spray each nostril) in healthy young adult men (n=16).

TABLE 4

| | | | AUC | | | |
|---|---|---|---|---|---|---|
| | | | (0, ∞)_{NS} | | | |
| | C_{max, NS} | t_{max, NS} | (ng · h · | t_{1/2, NS} | | |
| RIV | (ng · ml⁻¹) | (h) | ml⁻¹) | (h) | | FI, _{NS} |
| Mean | 13.8 [11.1] | 0.7 | 41.1 [33.9] | 2.94 | | 5.3 |
| Min-max | 3.1-49.0 | 0.3-1.5 | 8.7-106.8 | 1.3-7.9 | | 3.0-8.5 |
| CV (%) | 78 | 48 | 62 | | | 27 |
| 25^{th}, 75^{th}† | | | | 1.9, 4.4 | | |
| | | | AUC | | | |
| | | | (0, ∞)_{NS} | | | |
| NAP226- | C_{max, NS} | t_{max, NS} | (ng · h · | t_{1/2, NS} | | |
| 90 | (ng · ml⁻¹) | (h) | ml⁻¹) | (h) | N:R, _{NS} | FI, _{NS} |
| Mean | 3.0 [2.9] | 1.9 | 23.2 [22.7] | 4.4 | 0.55^{a} | 3.2 |
| Min-max | 0.8-3.0 | 1.0-3.0 | 15.4-34.7 | 3.2-6.9 | 0.27-2.48 | 2.4-4.3 |
| CV (%) | 27 | 36 | 22 | 21 | | 16 |
| 25^{th}, 75^{th}† | | | | | 0.45, 0.99 | |

Cmax, peak plasma concentration;
tmax, time of Cmax;
AUC(0, ∞), area under the plasma concentration-time curve from time zero to infinity;
t_{1/2}, terminal half-life;
FI, Fluctuation Index;
N:R, NAP226-90 AUC(0, ∞) to rivastigmine AUC(0, ∞) ratio;
NS, nasal;
^{a}median;
†25^{th} and 75^{th} percentiles;
[geometric mean].

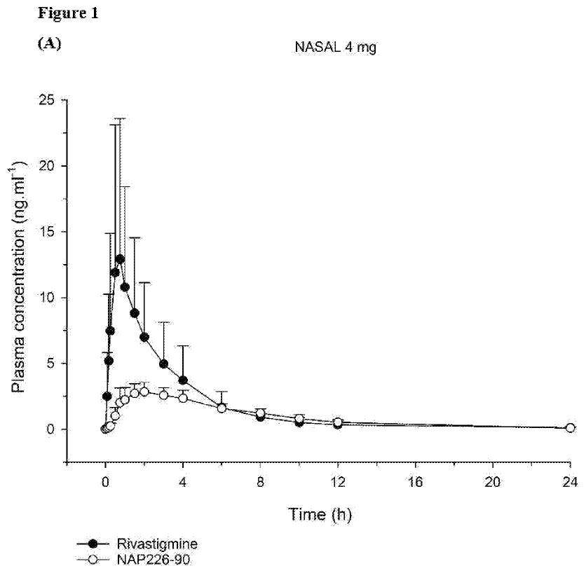

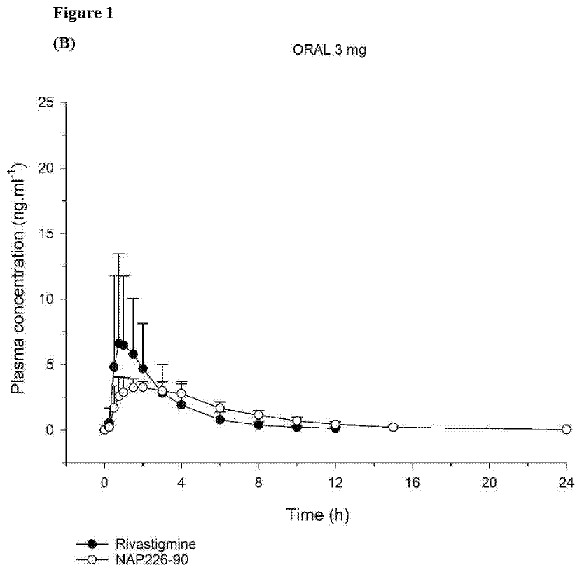

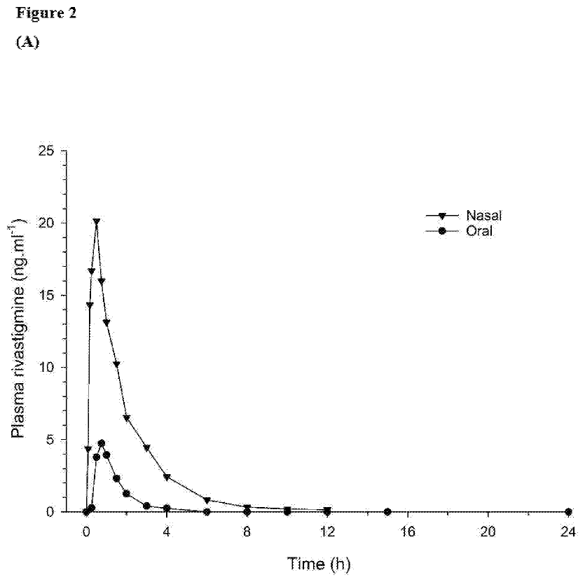

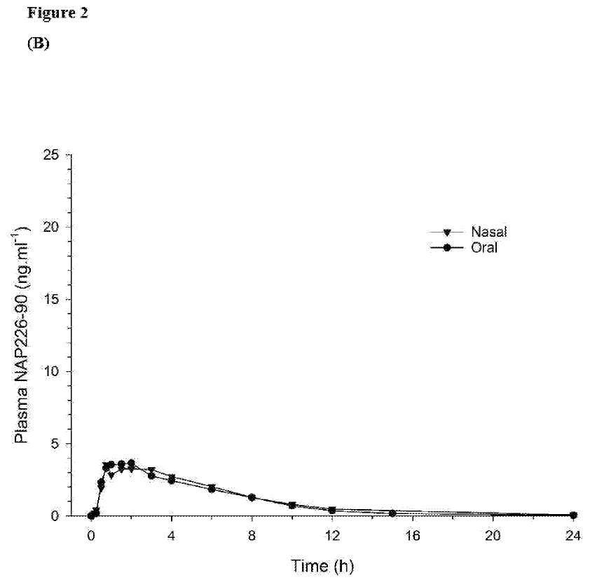

The initial clinical trial confirmed that rivastigmine, when administered as an intranasal formulation in accordance with the invention, was rapidly absorbed across the nasal mucosa with a mean (±SD) Tₘₐₓ of 0.7±0.3 h and a mean Cₘₐₓ of 13.8±10.8 ng/mL. In contrast, the absorption of rivastigmine via the oral route had a mean (±SD) Tₘₐₓ of 1.2±0.5 h and a mean Cₘₐₓ of 8.4±6.8 ng/mL. The extent of absorption of rivastigmine was clinically significant (FIGS. 1 and 2).

The median NAP226-90 to rivastigmine ratio for AUCO-∞ were 0.55 and 1.38, for the intranasal and oral treatments, respectively and these values were significantly different (p<0.001; Wilcoxon Signed Rank Test, n=16). The ratio for the intranasal treatment is comparable to that previously measured for the transdermal patch (0.67), and is advantageously 2.5-fold lower than that observed ratio for the oral capsule in this clinical study. It is generally recognised that a high degree of first-pass metabolism in the liver and gut after oral administration of rivastigmine results in much higher peripheral exposure to the metabolite (NAP226-90) (Polinsky 1998). The high low NAP226-90: rivastigmine AUC ratio observed for the intranasal sprays in accordance with the invention are indicative that nasal absorption is the dominant absorption pathway, and further suggests oral absorption due to nasal mucociliary clearance (Merkus et al. 1998) is limited.

Initial examination of single dose safety for rivastigmine intranasal sprays in accordance with the present invention indicate the solutions are safe to administer with limited or no side effects amongst participants who completed the clinical study as per protocol. In the group (n=16), two participants on intranasal treatment had treatment-emergent adverse events (one moderate nausea and one with mild headache) and three participants on oral treatment had treatment-emergent adverse events (one with mild nausea and two with mild dizziness). One participant vomited 34 minutes after oral dosing. This same participant did not vomit after intranasal dosing. This participant's pharmacokinetic data was prospectively excluded from the pharmacokinetic data set used for statistical analysis as per protocol.

Advantageously, low nausea (6%), and no vomiting or diarrhoea was observed with intranasal administration of rivastigmine despite the geometric mean rivastigmine AUCO-∞ being about double that observed for oral administration even after adjusting the intranasal value to an equivalent 3 mg dose (i.e., 33.9×3/4=25.4 ng.h/mL intranasal/13.2 ng.h/mL oral=1.9). Adverse gastrointestinal events (nausea, vomiting, diarrhea, weight loss and anorexia) have been significantly correlated with exposure to the metabolite NAP226-90 (both Cₘₐₓ and AUC) resulting from first-pass metabolism in the liver and gut after oral administration of rivastigmine. No such correlation is associated with exposure to rivastigmine itself in the absence of the metabolite (US FDA NDA No. 20-823, Spencer and Noble 1998). The observed reduction or elimination of adverse gastrointestinal side effects with intranasal administration of rivastigmine represents a significant advantage. It has been suggested that a low rivastigmine fluctuation index (FI) may reduce oral-related adverse gastrointestinal events by administration of rivastigmine via a transdermal patch (median FI, 0.7; Kurz et al. 2009; Lefevre et al. 2008). However, advantageously, low nausea (6%), and no vomiting and or diarrhoea adverse events were observed after intranasal administration even though the mean rivastigmine FI (5.3) after intranasal administration was significantly higher (p<0.005; paired t-test) than the mean rivastigmine FI (3.8) observed after oral administration.

The metered-dose intranasal spray has inherent capability to provide improved individual dosage adjustment within, below and above an effective dosing range. This may be beneficial in the case of OP poisoning, which may involve variations in rivastigmine dosage depending on a number of factors, including the degree of exposure and the severity of symptoms. A metered-dose intranasal spray as disclosed herein may advantageously provide improved individual dosage adjustment.

As indicated by the abovementioned clinical trial results, the present invention provides a method of intranasal rivastigmine use that approximately doubles the rivastigmine exposure of oral rivastigmine but with lower NAP226-90 exposure (i.e., peripheral cholinergic burden) in healthy young humans (Tables 1 and 2; FIGS. 1 and 2).

Without wishing to be bound by theory, the improvement in tolerability (i.e., reduction in side effects) of intranasal rivastigmine in healthy young humans may be explained by the reduction in the peripheral cholinergic burden caused by intranasal rivastigmine compared to oral rivastigmine. The reduction in peripheral cholinergic burden may be measured by a decrease in plasma NAP226-90 AUC to rivastigmine AUC ratio in healthy young adult humans.

It has surprisingly been found that the plasma NAP226-90 AUC to rivastigmine AUC ratio in healthy young adult humans may be significantly decreased with intranasal rivastigmine compared to oral rivastigmine, corresponding to improved tolerability. The improved tolerability observed with intranasal rivastigmine may be observed despite a high Cₘₐₓ and low tₘₐₓ for rivastigmine. This contrasts with the broadly accepted and published design rationale for transdermal rivastigmine, which aimed to use a low rivastigmive Cₘₐₓ and long tₘₐₓ in order to improve tolerability (Kurz et al., 2009; Lefèvre et al. 2008). However, this design rationale causes a detrimental loss of pharmacodynamic efficiency, whereby a much higher rivastigmine AUC exposure (approximately 60-70% higher) is needed for the same efficacy as higher Cₘₐₓ dosage forms, such as oral rivastigmine.

### Example 2

### Plasma BuChE and CSF AChE Enzyme Inhibition

A pre-treatment study with physostigmine transdermal patch treatment in rhesus monkeys showed protection against soman poisoning once plasma BuChE inhibition from this treatment reached approximately 40% (Cho et al., 2012). Physostigmine is a natural alkaloid that has poor tolerability in humans after oral administration. It is well known in the art that plasma BuChE enzyme inhibition by rivastigmine correlates with both brain (CSF) AChE and BuChE enzyme inhibition in humans (Cutler et al. 1998), and the individual pharmacodynamic treatment response afforded by intranasal rivastigmine can be readily monitored using a rapid, finger-prick blood-test for plasma BuChE or traditional pathology blood test.

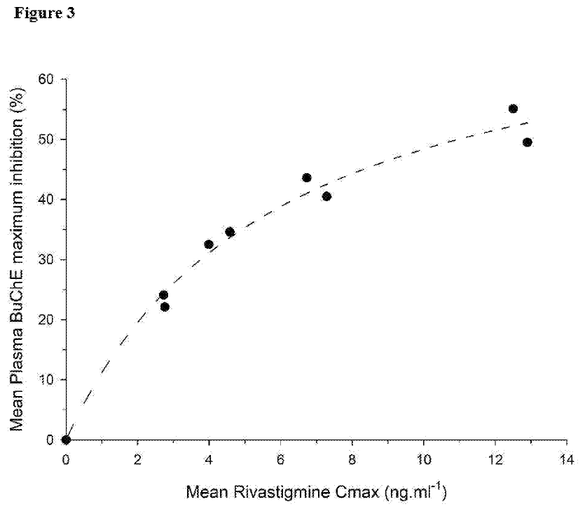

In healthy males, previous work by Lefèvre et al., 2009 was used to show a relationship between mean plasma rivastigmine Cₘₐₓ and the mean maximal inhibition % of plasma BuChE based upon Michaelis-Menten enzyme inhibition kinetics as estimated by the dotted line shown in FIG. 3. Based upon FIG. 3, an intranasal plasma rivastigmine Cₘₐₓ of at least 7.5 ng/ml would be expected to result in about 40% maximal inhibition of plasma BuChE in healthy males and be sufficient to afford CNS protection against OP poisoning.

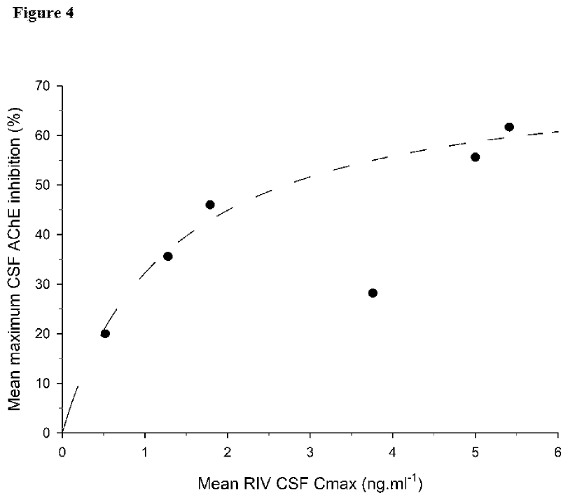

In Alzheimer's disease patients, previous work by Cutler et al., 1998 was used to show a relationship between mean CSF rivastigmine Cₘₐₓ and the mean maximal inhibition % of CSF AChE based upon Michaelis-Menten enzyme inhibition kinetics as estimated by the dotted line shown in FIG. 4. Based upon FIG. 4, an intranasal plasma rivastigmine Cₘₐₓ of at least 7.5 ng/ml would be expected to result in a CSF rivastigmine Cₘₐₓ of about 3 ng/mL based on the known rivastigmine plasma to CSF partition coefficient (PC) of 0.398 (i.e., 7.5×0.398=3; PC taken from Gobburu et al., 2001). This intranasal 3 ng/mL CSF rivastigmine Cₘₐₓ would be expected to result in about 50% maximal inhibition of CSF AChE in a human and be sufficient to afford CNS protection against OP poisoning.

Further, the AUC for BuChE or AChE % enzyme inhibition can be estimated from plasma rivastigmine concentration versus time profiles based upon the known relationships shown in FIGS. 3 and 4, and a plasma to CSF PC of 0.398. This parameter is useful for determining the average % enzyme inhibition attained over a period of time. For example, an intranasal AUC of about 40 ng.h/mL over about 12 hours would provide an average plasma BuChE inhibition of about 26% and an average CSF AChE inhibition of about 35%.

In contrast, oral galantamine partitions poorly from plasma to CSF in a human as previously shown by Nordberg et al., 2009. Nordberg et al. showed that an average 8.8 mg/d of oral rivastigmine over 13 weeks resulted in a decreased activity of 42.6% and 45.6% for AChE and BuChE in CSF, respectively, but an average 20.8 mg/d of oral galantamine only inhibited CSF AChE activities by 2.1% and CSF BuChE activities by 0.5% (both p <0.001 versus rivastigmine; p =not significant versus galantamine baseline).

Accordingly, compared to galantamine, the intranasal rivastigmine formulations disclosed herein may provide faster, more efficient, more reliable and prolonged inhibition of cholinesterase enzymes in the CNS to provide an improved treatment for OP poisoning. The intranasal rivastigmine formulations disclosed herein may also reduce side effects (e.g., nausea, vomiting, diarrhoea, headache) relative to existing methods, such as oral rivastigmine, oral galantamine and/or intramuscular galantamine.

### REFERENCES

Aracava Y, Pereira E F R, Akkerman M, Adler M, Albuquerque E X. Effectiveness of donepezil, rivastigmine, and (+/−)huperzine A in counteracting the acute toxicity of organophosphorus nerve agents: Comparison with galantamine J Pharmacol Exp Ther. 2009 331(3):1014-24.

Birks J, Grimley Evans J, Iakovidou V, Tsolaki M, Holt F E.Rivastigmine for Alzheimer's disease. Cochrane Database Syst Rev. 2009 Apr. 15; (2):CD001191.

Birks J S, Grimley Evans J. Rivastigmine for Alzheimer's disease. Cochrane Database Syst Rev 2015; 4:CD001191.

Buckley et al. Overcoming apathy in research on organophosphate poisoning. BMJ. 2004. 329:1231-1233.

Chen J, Pan H, Chen C, Wu W, Iskandar K, He J, Piermartiri T, Jacobowitz DM, Yu Q S, McDonough J H, Greig N H, Marini A M. (−)-Phenserine attenuates soman-induced neuropathology. 2014 PLOS ONE 9(6): e99818.

Cutler N R, Polinsky R J, Sramek J J, Enz A, Jhee S S, Mancione L, Hourani J, Zolnouni P. Dose-dependent CSF acetylcholinesterase inhibition by SDZ ENA 713 in Alzheimer's disease. Acta Neurol Scand 1998; 97:244-50.

Feldman H H, Lane R; Study 304 Group. Rivastigmine: a placebo controlled trial of twice daily and three times daily regimens in patients with Alzheimer's disease. J Neurol Neurosurg Psychiatry. 2007 October; 78(10):1056-63.

Gobburu J V, Tammara V, Lesko L, Jhee S S, Sramek J J, Cutler N R, Yuan R. Pharmacokinetic-pharmacodynamic modeling of rivastigmine, a cholinesterase inhibitor, in patients with Alzheimer's disease. J Clin Pharmacol. 2001 October; 41(10):1082-90.

Grossberg G T, Sadowsky C, Olin J T. Rivastigmine transdermal system for the treatment of mild to moderate Alzheimer's disease. Int J Clin Pract. 2010 April; 64(5):651-60.

Hossain M, Jhee S S, Shiovitz T, McDonald C, Sedek G, Pommier F, Cutler N R. Estimation of the absolute bioavailability of rivastigmine in patients with mild to moderate dementia of the Alzheimer's type. Clin Pharmacok/net. 2002; 41(3):225-34.

Kadir A, Darreh-Shori T, Almkvist O, Wall A, Grut M, Strandberg B, Ringheim A, Eriksson B, Blomquist G, Langstrom B, Nordberg A. PET imaging of the in vivo brain acetylcholinesterase activity and nicotine binding in galantamine-treated patients with AD. Neurobiol Aging 2008 29(8):1204-17.

Kurz A, Farlow M, Lefevre G. Pharmacokinetics of a novel transdermal rivastigmine patch for the treatment of Alzheimer's disease: a review. Int J Clin Pract. 2009 May; 63(5):799-805.

Lane M, Carter D, Pescrille J D, Aracava Y, Fawcett W P, Basinger G W, Pereira E F R, Albuquerque E X. Oral Pretreatment with Galantamine Effectively Mitigates the Acute Toxicity of a Supralethal Dose of Soman in Cynomolgus Monkeys Posttreated with Conventional Antidotes. J Pharmacol Exp Ther. 2020 375(1):115-126.Larner A J. Transdermal rivastigmine for Alzheimer's disease: skin deep or scratching the surface? Int J Clin Pract. 2010 April; 64(5):534-6.

Lavon O, Eisenkraft A, Blanca M, Raveh L, Ramaty E, Krivoy A, Atsmon J, Grauer E, Brandeis R. Is rivastigmine safe as pretreatment against nerve agents poisoning? A pharmacological, physiological and cognitive assessment in healthy young adult volunteers. Neurotoxicology 2015; 49: 36-44.

Lefevre G, Sedek G, Jhee S S, Leibowitz M T, Huang H L, Enz A, Maton S, Ereshefsky L, Pommier F, Schmidli H, Appel-Dingemanse S. Pharmacokinetics and pharmacodynamics of the novel daily rivastigmine transdermal patch compared with twice-daily capsules in Alzheimer's disease patients. Clin Pharmacol Ther 2008; 83:106-14.

Lefevre G, Biiche M, Sedek G, Maton S, Enz A, Lorch U, Sagan C, Appel-Dingemanse S. Similar rivastigmine pharmacokinetics and pharmacodynamics in japanese and white healthy participants following the application of novel rivastigmine patch. J Clin Pharmacol 2009 49(4):430-43.Maidment I, Fox C, Boustani M. Cholinesterase inhibitors for Parkinson's disease dementia. Cochrane Database Syst Rev 2006; 1:CD004747.

Merkus F W, Verhoef J C, Schipper N G, Marttin E. Nasal mucociliary clearance as a factor in nasal drug delivery. Adv Drug Deliv Rev 1998 29:13-38.

Morgan T M, Soh B. Absolute bioavailability and safety of a novel rivastigmine nasal spray in healthy elderly individuals. Br J Clin Pharmacol 2017; 83: 510-516.

Nordberg A, Darreh-Shori T, Peskind E, Soininen E, Mousavi M, Eagle G, Lane R. Different cholinesterase inhibitor effects on CSF cholinesterases in Alzheimer patients. Curr Alzheimer Res 2009 6(1):4-14.Polinsky RI. Clinical pharmacology of rivastigmine: a new-generation acetylcholinesterase inhibitor for the treatment of Alzheimer's disease. Clin Ther 1998; 20:634-47.

Remington's Pharmaceutical Sciences, Sixteenth Edition, E. W. Martin, Mack Publishing Co., Easton, Pa., 1980.

Remington: The Science and Practice of Pharmacy, 21st Edition, Lippincott Williams & Wilkins, 2005

Sidell F R, Newmark J, McDonough J H. Nerve agents. In: Tuorinsky S D, editor. Medical aspects of chemical warfare. Washington, DC: Office of the Surgeon General, US, Army, Borden Institute, Walter Reed Army Medical Center; 2008, pp. 155-220.

Spencer C M, Noble S. Rivastigmine. A review of its use in Alzheimer's disease. Drugs Aging 1998; 13:391-411.

Tariot P N, Solomon P R, Morris J C, Kershaw P, Lilienfeld S, Ding C. A 5-month, randomized, placebo-controlled trial of galantamine in AD. The Galantamine USA-10 Study Group. Neurology. 2000 Jun. 27; 54(12):2269-76

United States Food and Drug Administration, website: www.fda.gov. US FDA NDA Applic. No. 20-823. Clinical pharmacology and biopharmaceutics review(s). Rivastigmine tartrate capsules. p.vii, p.xi., p.xiv. File accessed from Drugs@FDA on 14 September

Wilcock G K, Lilienfeld S, Gaens E on behalf of the galantamine international-1 study group. Efficacy and safety of galantamine in patients with mild to moderate Alzheimer's disease: multicentre randomised controlled trial. BMJ 2000 321:1-7.

Williams A C, Barry BW. Terpenes and the lipid-protein-partitioning theory of skin penetration enhancement. Pharm Res. 1991 Jan; 8(1):17-24.

Winblad B, Cummings J, Andreasen N, Grossberg G, Onofrj M, Sadowsky C, Zechner S, Nagel J, Lane R. A six-month double-blind, randomized, placebo-controlled study of a transdermal patch in Alzheimer's disease--rivastigmine patch versus capsule. Int J Geriatr Psychiatry 2007; 22:456-67.

1. A method for treating or preventing organophosphorus poisoning in a subject comprising administering an effective amount of a sustained-release aqueous intranasal formulation to a subject, wherein the formulation comprises rivastigmine or a pharmaceutically acceptable salt or solvate thereof, a pH modifying agent and a thickening agent, wherein:
the rivastigmine comprises about 0.5% to about 15% by weight of the total formulation;
the thickening agent comprises about 0.25% to about 2% by weight of the total formulation; and
the pH of the formulation is in the range of about 3 to 6.

2. The method according to claim 1, wherein the organophosphorus poisoning is caused by exposure of the subject to an organophosphorus nerve agent or an organophosphorus pesticide.

3. The method according to claim 2, wherein the organophosphorus nerve agent is selected from the group consisting of: sarin (2-(fluoro-methylphosphoryl)oxypropane), cyclosarin ([fluoro(methyl)phosphoryl]oxycyclohexane), soman (3-(fluoro-methyl-phosphoryl)oxy-2,2-dimethyl-butane), VR (N,N-diethyl-2-(methyl-(2-methylpropoxy)phosphoryhsulfanylethanamine), VX (S-2-[diisopropylamino]O-ethyl methylphosphonothioate), tabun (ethyl N,N-dimethylphosphoramidocyanidate), Novichok agents, and combinations thereof.

4. The method according to claim 2, wherein the organophosphorus pesticide is selected from the group consisting of: parathion, fenthion, malathion, diazinon, dursban, chlorpyrifos, terbufos, acephate, phorate, methyl parathion, phosmet, azinphos-methyl, dimethoate, and combinations thereof.

5. The method according to claim 1, wherein the subject is a human.

6. The method according to claim 5, wherein the human is a healthy adult.

7. The method according to claim 6, wherein the human is a healthy young adult.

8. The method according to claim 1, wherein the rivastigmine is rivastigmine free base or rivastigmine tartrate.

9. The method according to claim 1, wherein the pH modifying agent is selected from citrate buffer and citric acid, and comprises about 0.01% to about 2% by weight of the total formulation.

10. The method according to claim Jany one of claims 1, wherein the thickening agent is selected from the group consisting of methylcellulose, ethylcellulose, hydroxy-ethylcellulose, hydroxyl propyl cellulose, hydroxy propyl methylcellulose, sodium carboxy methylcellulose, polyacrylic acid polymers, poly hydroxyethyl methylacrylate, polyethylene oxide, polyvinyl pyrrolidone, polyvinyl alcohol, tragacanth, sodium alginate, guar gum, xanthan gum, lectin, soluble starch, gelatin, pectin, chitosan, and combinations thereof.

11. The method according to claim 1, wherein the formulation further comprises a sensory agent selected from a C₂ to C₄ alcohol, menthols, terpenes, thymol, camphor, capsicum, phenol, carveol, menthol glucuronide, eucalyptus oil, benzyl alcohol, salicyl alcohol, ethanol, isopropanol, clove bud oil, mint, spearmint, peppermint, eucalyptus, lavender, citrus, lemon, lime, hexylresorcinol, ketals, diols, and mixtures thereof.

12. The method according to claim 11, wherein the sensory agent comprises about 1% to about 15% by weight of the total formulation.

13. The method according to claim 1, further comprising administering an additional therapeutic agent selected from atropine, pralidoxime, a benzodiazepine, pyridostigmine, galantamine, and combinations thereof to the subject.

14. The method according to claim 1, wherein the rivastigmine or pharmaceutically acceptable salt thereof is administered in an amount equivalent to up to 16 mg rivastigmine free base per day.

15. The method according to claim 1, wherein the rivastigmine or pharmaceutically acceptable salt thereof is administered in an amount equivalent to up to 12 mg rivastigmine free base per day.

16. The method according to claim 1, wherein the rivastigmine or pharmaceutically acceptable salt thereof is administered in an amount equivalent to up to 4 mg rivastigmine free base per day.

17. The method according to claim 1, wherein, following intranasal administration of an effective amount to a subject, the maximum rivastigmine plasma concentration (Cₘₐₓ) of the subject is at least about 7.5 ng/mL.

18. The method according to claim 1, wherein, following intranasal administration of an effective amount to a subject, the maximum rivastigmine plasma concentration (Cₘₐₓ) of the subject is at least about 14 ng/mL.

19. The method according to claim 1, wherein, following intranasal administration of an effective amount to a subject, the maximum rivastigmine plasma concentration is achieved within about 3 hours (Tₘₐₓ).

20. The method according to claim 1, wherein, following intranasal administration of an effective amount to a subject, the maximum rivastigmine plasma concentration is achieved within about 1 hour (Tₘₐₓ).

21. The method according to claim 1, wherein, following intranasal administration of an effective amount to a subject, the plasma NAP226-90 AUC to rivastigmine AUC ratio is less than 1.

22. The method according to claim 1, wherein, following intranasal administration of an effective amount to a subject, the plasma NAP226-90 AUC to rivastigmine AUC ratio is less than 0.6.

23. The method of claim 1, wherein the average AUC is greater than 10 ng.h per ml per mg dose of rivastigmine.

24. The method according claim 1, wherein the intranasal administration is associated with a reduced incidence of side effects compared to oral administration.

25. Use of a sustained-release aqueous intranasal formulation in the preparation of a medicament for treating or preventing organophosphorus poisoning, wherein the treating or preventing comprises intranasal administration of the medicament to a subject, and wherein the formulation comprises rivastigmine or a pharmaceutically acceptable salt or solvate thereof, a pH modifying agent and a thickening agent, wherein:
the rivastigmine comprises about 0.5% to about 15% by weight of the total formulation;
the thickening agent comprises about 0.25% to about 2% by weight of the total formulation; and
the pH of the formulation is in the range of about 3 to 6.
